(19) United States
(12) Patent Application Publication
(10) Pub. No.: US 2015/0225800 A1
(43) Pub. Date: Aug. 13, 2015

(54) METHODS AND ASSAYS FOR TREATMENT OF BLADDER CANCER

(71) Applicant: THE GENERAL HOSPITAL CORPORATION, Boston, MA (US)

(72) Inventors: Xuanhui Sharron LIN, Newton, MA (US); William Scott MCDOUGAL, Manchester, MA (US); Chin-Lee WU, Newton, MA (US)

(73) Assignee: THE GENERAL HOSPITAL CORPORATION, Boston, MA (US)

(21) Appl. No.: 14/698,254

(22) Filed: Apr. 28, 2015

Related U.S. Application Data
(63) Continuation-in-part of application No. PCT/US2013/067792, filed on Oct. 31, 2013.
(60) Provisional application No. 61/795,990, filed on Oct. 31, 2012.
(60) Provisional application No. 61/721,300, filed on Nov. 1, 2012.

(51) Int. Cl. C12Q 1/68

(52) U.S. Cl. CPC C12Q 1/6886; C12Q 2600/158; C12Q 2600/16

(57) ABSTRACT

The technology described herein relates to methods of prognosing and treating bladder cancer.

### CROSS-REFERENCE TO RELATED APPLICATIONS

This application is a continuation-in-part application of International Patent Application No. PCT/US13/067792 filed Oct. 31, 2013, which designates the United States and claims benefit under 35 U.S.C. §119(e) of U.S. Provisional Applications No. 61/795,990 filed Oct. 31, 2012 and No. 61/721,300 filed Nov. 1, 2012, the contents of which are incorporated herein by reference in their entirety.

### TECHNICAL FIELD

The invention relates to methods and systems of treating and prognosising bladder cancer.

### BACKGROUND

Bladder cancer treatments vary significantly depending upon whether the cancer is or will become malignant. Subjects who have non-invasive cancer are best treated by conservative methods, while the more aggressive approach of cystectomy (removal of the bladder) and/or systemic chemotherapy/radiation is necessary for subjects with invasive cancers. About 80% of the new diagnoses of bladder cancer involve subjects with non invasive cancer, many of which will eventually experience a transtition to invasive disease and potentially metastatic cancer. With current technologies available in the clinic, it is not possible to accurately predict which patients will develop invasive or metastatic cancer, and thus numerous subjects do not receive the optimal treatment for their particular condition.

### SUMMARY

As described herein, the inventors have identified gene signatures which permit the identification of patients who will benefit from (e.g. have optimal outcomes) cystectomy and/or chemotherapyas treatment for bladder cancer. Accordingly, provided herein are methods of treatment and assays relating to bladder cancer and the administration of cystectomies.

In one aspect, described herein is a method of treatment comprising, detecting, in a sample obtained from a subject in need of treatment for bladder cancer, the level of expression products of at least one marker gene selected from Table 1 or Table 2; administering cystectomy or chemotherapy to the subject if the level of expression products selected from Table 1 are increased relative to a reference level or the level of expression products selected from Table 2 are decreased relative to a reference level; and not administering a cystectomy or other invasive treatment to the subject if the level of expression products selected from Table 1 are not increased relative to a reference level or the level of expression products selected from Table 2 are not decreased relative to a reference level. In one aspect, described herein is a method of treatment comprising, administering cystectomy or chemotherapy to a subject determined to have a level of expression products selected from Table 1 increased relative to a reference level or a level of expression products selected from Table 2 decreased relative to a reference level; and not administering a cystectomy or other invasive treatment to a subject determined to have a level of expression products selected from Table 1 not increased relative to a reference level or a level of expression products selected from Table 2 not decreased relative to a reference level.

In one aspect, described herein is an assay comprising, detecting, in a sample obtained from a subject in need of treatment for bladder cancer, the level of expression products of at least one marker gene selected from Table 1 or Table 2; wherein the subject is likely to benefit from cystectomy or chemotherapy if the level of expression products selected from Table 1 is increased relative to a reference level or the level of expression products selected from Table 2 is decreased relative to a reference level; and wherein the subject is not likely to benefit from a cystectomy or other invasive treatment if the level of expression products selected from Table 1 is not increased relative to a reference level or the level of expression products selected from Table 2 is not decreased relative to a reference level.

In one aspect, described herein is a method of determing if a subject is likely to benefit from cystectomy, the method comprising, detecting, in a sample obtained from a subject in need of treatment for bladder cancer, the level of expression products of at least one marker gene selected from Table 1 or Table 2; wherein the subject is likely to benefit from cystectomy or chemotherapy if the level of expression products selected from Table 1 is increased relative to a reference level or the level of expression products selected from Table 2 is decreased relative to a reference level; and wherein the subject is not likely to benefit from a cystectomy or other invasive treatment if the level of expression products selected from Table 1 is not increased relative to a reference level or the level of expression products selected from Table 2 is not decreased relative to a reference level.

In some embodiments, the one or more marker genes is selected from the genes of Table 3. In some embodiments, the one or more marker genes is selected from the group consisting of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In some embodiments, the level of the expression products of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5 are determined In some embodiments, the expression products are mRNA expression products. In some embodiments, the expression products are polypeptide expression products. In some embodiments, the subject has TA or T1 bladder cancer. In some embodiments, the sample is a tumor cell sample. In some embodiments, the sample is a urine sample. In some embodiments, the subject is a human.

### DESCRIPTION OF THE DRAWINGS

FIG. 1 depicts a schematic of the study design and RNA-Seq analysis workflow.

FIG. 2 depicts a graph of the sample clustering analysis of the significantly differentially expressed genes between 3 T1NP and 4 T1P samples.Hierarchical cluster analysis of RNA-Seq data profiling bladder tissue obtained from 3 T1NP patients of know BCa with non-progressive status and 4 T1P BCa with known muscle invasive progressive disease, first diagnosed as non-progressive (T1) and then progressed to T>=2 progressive BC. There were a total of 181 genes significantly differentially expressed between 3 T1NP and 4 T1P samples with p<0.05. The hierarchical clustering dendrogram from data obtained from the 4 T1P samples was plotted in regard the time of for T1 progression to muscle invasive tumor. For patients with BLK21, BLK20, BLK24 and BLK34, it took 4.5, 1.4, 0.8 and 3 years for their disease to progress from T1 to T2 (muscle invasive) BCa. Y-axis indicates the time (years) for the progression from T1 to muscle invasive BCa.

FIG. 3 depicts a schematic of the top enriched network associated with BCa Progression. Map was created using Ingenuity Pathway Analysis™ (IPA) software. Lines represent reported interactions between proteins.

FIGS. 4A-4B demonstrate the overlap of gene lists between RNA-Seq and DASL Cancer Panel platforms. FIG. 4A depicts a Venn diagram of the overlap between 181 significantly differentially expressed genes from RNA-Seq platform (181) and the 502 DASL Cancer Panel genes (502). Of the 181 differentially expressed genes from RNA-Seq analysis, 13 genes were found in the 502 DASL Cancer Panel. FIG. 4B depicts a Venn diagram of the 13 genes significantly differential expressed from the RNA-Seq analysis (13) and the 26 significantly differentially expressed genes from DASL Cancer Panel platform. Note that five genes are differentially expressed in both analyses. See Table 8 for more details of the five common genes.

### DETAILED DESCRIPTION

As described herein, the inventors have discovered that a number of genes are differentially regulated in tumors of patients with progressive bladder cancer who will benefit from cystectomy and/or chemotherapyas compared to those subjects with non-progressive bladder cancer who will not benefit from cytoreductive surgery. Accordingly, there are provided herein methods and assays relating to the prognosis, risk assessment, and treatment of subjects having bladder cancer, particularly as relates to subjects receiving cystectomy and/or chemotherapyas part of their treatment regimen for bladder cancer.

“Bladder cancer” refers to cancers arising in, or involving, the bladder, e.g. in the epithelium of the bladder. As used herein, the term “cancer”or “tumor” refers to an uncontrolled growth of cells which interferes with the normal functioning of the bodily organs and systems. A subject who has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs.

In some embodiments, the methods described herein relate to treating a subject having or diagnosed as having bladder cancer. Subjects having bladder cancer can be identified by a physician using current methods of diagnosing bladder cancer. Symptoms and/or complications of bladder cancer which characterize these conditions and aid in diagnosis are well known in the art and include but are not limited to hematuria, and painful and/or frequent urination. Tests that may aid in a diagnosis of, e.g. bladder cancer include, but are not limited to, cystoscopy, NMP22, human complement factor H-related protein, carcinoembryonic antigen, FGFR3, CERTNDX™, UROVYSION™, CXBLADDER™, HEXVIX™/CYSVIEW™, and transurethral resection tests. A family history of cancer or exposure to risk factors for bladder cancer (e.g. smoking) can also aid in determining if a subject is likely to have bladder cancer or in making a diagnosis of bladder cancer.

The standard treatment for bladder cancer varies significantly depending upon the stage and/or severity of the bladder cancer. Subjects with TA or T1 cancer are typically treated by local surgical removal of the diseased tissue and/or partial resection. Subjects with T2 (or greater, e.g. T3 or T4) cancer are typically treated by cystectomy, i.e. removal of the bladder, and optionally additional internal organs. Subjects with T2 (or greater) cancer can also be treated with chemotherapy and/or radiation, and option not typically persued for TA or T1 cancer patients. Of subjects diagnosed with TA and/or T1 cancer, some have cancers that will progress to T2 and other have cancers that will not progress to T2 (or greater, e.g. T3 or T4). Clinical outcomes can be greatly improved by identifying which subjects have progressive and/or potentially progressive cancers and treating them with a cystectomy or chemotherapy before the cancer actually progresses to the T2 stage (or greater, e.g. T3 or T4). In some embodiments, “benefiting from cystectomy or chemotherapy” can refer to a subject who will have an optimal outcome from cystectomy or chemotherapy, e.g. particularly an early treatment before the cancer reaches a T2 stage (or greater, e.g. T3 or T4).

Conversely, if a subject has a TA or T1 bladder cancer that will not progress to a T2 (or greater) bladder cancer, then invasive treatments are unnecessary for that patient. Accordingly, provided herein are methods relating to diagnosing, prognosing, and treating subjects with bladder cancer, comprising not administering an invasive treatment to a subject determined to have a non-progressive cancer according to the methods and assays described below herein. An invasive treatment for bladder cancer can include cystectomy, chemotherapy (including radiation therapy), radical TUR, or segmented partial resection. Removal of tumors, standard TUR, and/or partial resection are not considered invasive treatmens as defined herein.

As described herein, the inventors have identified certain genes which are differentially regulated, to a statistically significant degree, as compared to a reference level, in subjects who have progressive TA or T1 bladder cancer (i.e. bladder cancer that will, absent cystectomy, progress to a T2 cancer). These subjects will benefit from cystectomy or chemotherapy, particularly early treatment before the cancer progresses to T2. The identified genes are sometimes referred to herein as marker genes to indicate their relation to being a marker for whether cystectomy and/or chemotherapy will be efficacious. Accordingly, some embodiments of the invention are generally related to assays, methods and systems for assessing the likely response of a subject to cystectomy and/or chemotherapy and/or radiation and/or treating a subject for bladder cancer, e.g. by determining if a subject will benefit from cystectomy and/or chemotherapy and/or radiation and administering the treatment if it is likely to be efficacious. In certain embodiments, the assays and methods are directed to determination and/or measurement of the expression level of a gene product (e.g. protein and/or gene transcript such as mRNA) in a biological sample of a subject. In certain embodiments the assays and methods are directed to determination of the expression level of a gene product of at least two genes in a biological sample of a subject, i.e. at least two genes, at least three genes, at least four genes , at least five genes, at least six genes, at least seven genes, at least eight genes, at least nine genes, at least 10 genes . . . at least 15 genes, . . . at least 25 genes, . . . at least 30 genes, or more genes, or any number of genes selected from Table 1 and/or Table 2 as described herein. In some embodiments, the marker gene(s) is selected from the group listed in Table 3. In some embodiments, the marker gene(s) is selected from the group consisting of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In some embodiments, the assays, methods, and systems described herein are directed to determination of the expression level of a gene product of at least two genes in a biological sample of a subject, e.g. at least two genes, or at least three genes, or at least four genes, or, e.g. all of the following genes: IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5.

TABLE 1
Genes upregulated in progressive bladder cancers
as compared to non-progressive bladder cancers

| SYMBOL | logFC(P/NP) | t | P. Value |
|---|---|---|---|
| IGFBP5 | 5.28378713 | 6.394460723 | 0.000213397 |
| 5S_rRNA | 5.02822492 | 3.43620067 | 0.00891841 |
| ANXA1 | 4.20673169 | 2.74521567 | 0.02532413 |
| LSP1 | 4.17187565 | 5.47618297 | 0.00059692 |
| CD44 | 4.002931343 | 2.898246336 | 0.020015917 |
| COL7A1 | 3.97001825 | 2.81379523 | 0.02278522 |
| F3 | 3.9258451 | 3.15268785 | 0.013601 |
| CSPG2 | 3.90783955 | 2.73594918 | 0.025688889 |
| NLRP1 | 3.72742532 | 3.64172336 | 0.00661003 |
| AEBP1 | 3.47639365 | 2.31948691 | 0.04906252 |
| STIM1 | 3.42021253 | 4.6546381 | 0.00164936 |
| CEP164 | 3.29752777 | 3.00803891 | 0.01692864 |
| CCND1 | 3.259008872 | 2.772126521 | 0.024294756 |
| UGT1A8 | 2.84921163 | 2.45825555 | 0.03952185 |
| ITGA2 | 2.80098403 | 3.51115212 | 0.00799032 |
| C9orf84 | 2.74245395 | 2.58125402 | 0.03264157 |
| CRISPLD2 | 2.73733092 | 3.0351325 | 0.01624599 |
| TBC1D4 | 2.7293094 | 3.93057252 | 0.00438191 |
| AGPS | 2.69481996 | 3.68793759 | 0.00618436 |
| LTBP1 | 2.65654311 | 3.4787794 | 0.00837794 |
| CASP8 | 2.615453795 | 3.415985067 | 0.009187841 |
| HEG1 | 2.58447166 | 2.46051813 | 0.03938287 |
| HLA-K | 2.54764192 | 3.2147809 | 0.01239004 |
| MAPKAPK3 | 2.45053475 | 2.64051867 | 0.02977474 |
| C20orf194 | 2.38897783 | 2.6786851 | 0.02806606 |
| SAMD9L | 2.37949244 | 2.65727264 | 0.02901193 |
| GPC1 | 2.37854774 | 2.703837 | 0.02699529 |
| RAB31 | 2.35711979 | 3.37006139 | 0.00983245 |
| DHRS7 | 2.27969466 | 3.46232681 | 0.00858254 |
| BIRC2 | 2.272732375 | 2.321656288 | 0.048896876 |
| ANTXR2 | 2.25454352 | 4.232024 | 0.00289011 |
| ACVR1 | 2.2424225 | 3.24529738 | 0.01183696 |
| ANXA7 | 2.19296609 | 3.32472129 | 0.01051596 |
| OPTN | 2.1810596 | 3.8899461 | 0.00463941 |
| SNX14 | 2.17884405 | 3.10806374 | 0.01454759 |
| DST | 2.17427338 | 2.53633429 | 0.03500086 |
| CCPG1 | 2.17244809 | 4.39155853 | 0.00233137 |
| FAM46A | 2.04425942 | 2.88029669 | 0.02057383 |
| PON2 | 2.03283625 | 2.82635351 | 0.02234953 |
| SH3BGRL | 2.00979907 | 2.48497519 | 0.03791168 |
| LY75 | 1.98260955 | 3.0894357 | 0.01496292 |
| FAM117B | 1.96932966 | 2.42171139 | 0.0418366 |
| RNF145 | 1.92196075 | 3.16552089 | 0.01334091 |
| DPYSL3 | 1.91576424 | 2.46411223 | 0.0391631 |
| SH3D19 | 1.91148603 | 3.54823504 | 0.00756958 |
| TINF2 | 1.89082248 | 2.74315871 | 0.02540463 |
| DCBLD1 | 1.87559116 | 2.52153527 | 0.03581555 |
| AMD1 | 1.87228868 | 3.15737632 | 0.01350536 |
| MAN2A1 | 1.85352136 | 3.14150199 | 0.01383205 |
| NFIA | 1.83804614 | 3.60419817 | 0.00697863 |
| SIPA1L2 | 1.83061168 | 3.02583092 | 0.01647705 |
| RNF141 | 1.82400796 | 2.64865981 | 0.02940154 |
| DSCR1 | 1.82239402 | 2.57040759 | 0.033196 |
| SKAP2 | 1.8216168 | 2.54941038 | 0.0342967 |
| LFNG | 1.81565969 | 2.33377226 | 0.047982 |
| SLC12A6 | 1.79124672 | 2.66045049 | 0.02886951 |
| PPFIBP1 | 1.78992859 | 2.87958916 | 0.02059615 |
| GNPDA1 | 1.76249261 | 2.93374338 | 0.01895834 |
| PAPSS1 | 1.73903819 | 2.97560137 | 0.01778546 |
| FARP2 | 1.72569239 | 2.60722749 | 0.03135196 |
| BTN3A3 | 1.72513362 | 2.76290781 | 0.02464244 |
| HMGN3 | 1.72280555 | 2.8250367 | 0.02239481 |
| C22orf9 | 1.72135934 | 2.48644227 | 0.03782522 |
| PDLIM5 | 1.70827373 | 3.39620038 | 0.00945989 |
| REEP3 | 1.69877498 | 2.56218205 | 0.03362284 |
| SETD7 | 1.66851341 | 2.41554395 | 0.04224053 |
| TAF10 | 1.65721124 | 2.75933465 | 0.02477858 |
| PNMA1 | 1.64661222 | 2.42756911 | 0.04145656 |
| TOR1B | 1.64511904 | 3.05763692 | 0.01570084 |
| FKBP3 | 1.64284734 | 2.54657645 | 0.03444808 |
| NUPL2 | 1.62895735 | 2.46787748 | 0.0389342 |
| ZNF330 | 1.62390269 | 2.35978476 | 0.04607531 |
| PSEN1 | 1.61493392 | 2.64427886 | 0.02960177 |
| MAP3K7IP2 | 1.59637386 | 2.55986085 | 0.0337443 |
| AHCTF1 | 1.57422991 | 2.53263207 | 0.03520289 |
| AUH | 1.56600683 | 3.25817799 | 0.01161137 |
| PAK1 | 1.54780871 | 2.66362608 | 0.02872791 |
| RPL36AL | 1.54280083 | 2.51151484 | 0.03637809 |
| FAM179B | 1.53609648 | 2.40545269 | 0.04290994 |
| TCIRG1 | 1.51981913 | 3.07851256 | 0.01521223 |
| IL6ST | 1.49181891 | 2.41008791 | 0.04260114 |
| CCDC109A | 1.48607943 | 2.38252992 | 0.04447048 |
| ESD | 1.47925646 | 2.35183634 | 0.04664972 |
| SLC35D1 | 1.46601041 | 2.77742706 | 0.02409714 |
| MGST2 | 1.46207904 | 2.47256346 | 0.03865121 |
| C19orf33 | 1.44051512 | 2.79278485 | 0.02353387 |
| SLC25A24 | 1.43315867 | 2.5446134 | 0.03455334 |
| CPSF3 | 1.4330224 | 2.31924323 | 0.04908116 |
| MARCH5 | 1.42197835 | 2.33767596 | 0.0476909 |
| PLSCR3 | 1.42090337 | 2.51142349 | 0.03638326 |
| NUMB | 1.39456495 | 2.72249068 | 0.02622831 |
| CYLD | 1.37478347 | 2.4480552 | 0.04015463 |
| RPS23 | 1.36488075 | 2.31757051 | 0.04920931 |
| C4orf34 | 1.34586324 | 2.41143698 | 0.04251169 |
| FBXL5 | 1.34532622 | 2.60776818 | 0.03132567 |
| PPP2R5C | 1.30705076 | 2.5104409 | 0.03643891 |
| MAP3K7 | 1.28229399 | 2.47940657 | 0.03824169 |
| CD164 | 1.26391916 | 2.33702462 | 0.04773935 |
| PTEN | 1.251488632 | 2.408825886 | 0.042684997 |
| ZBTB4 | 1.21710635 | 2.46352004 | 0.03919922 |
| UBE2D3 | 1.17089005 | 2.42638647 | 0.04153301 |

TABLE 2
Genes downregulated in progressive bladder cancers
as compared to non-progressive bladder cancers

| SYMBOL | logFC(P/NP) | t | P. Value |
|---|---|---|---|
| PLA2G2F | −4.156008 | −3.5080194 | 0.00802698 |
| C10orf76 | −3.8186756 | −4.2146327 | 0.00295927 |
| KALRN | −3.7281337 | −3.5440781 | 0.00761555 |
| CYP4B1 | −3.6225716 | −3.7176789 | 0.00592596 |
| CACNA1D | −3.5523114 | −2.3719126 | 0.0452125 |
| APOL4 | −3.2213341 | −4.6396991 | 0.00168164 |
| CYP4Z2P | −3.0769814 | −3.8493161 | 0.00491323 |
| ALDH16A1 | −2.8626969 | −3.5054908 | 0.00805671 |
| CRAT | −2.7553643 | −2.4480144 | 0.04015718 |
| UGCGL2 | −2.6472916 | −2.5068012 | 0.0366458 |
| ZNF704 | −2.6386071 | −2.4246841 | 0.0416433 |
| PICK1 | −2.627548 | −3.2740923 | 0.01133888 |
| POLE | −2.6239919 | −2.8515353 | 0.02150164 |
| FAM73B | −2.5974501 | −2.596323 | 0.03188693 |
| CAMK2N1 | −2.5201877 | −3.2934832 | 0.01101595 |
| GATA2 | −2.5116284 | −2.6183502 | 0.03081572 |
| PTGR1 | −2.4453105 | −3.0447173 | 0.01601143 |
| VEGF | −2.408409244 | −3.748738239 | 0.00566838 |
| SLC19A2 | −2.4081763 | −3.1417375 | 0.01382714 |
| ABCA5 | −2.3816459 | −4.0291014 | 0.003819 |
| TRAF4 | −2.350257868 | −3.381925006 | 0.00966147 |
| CGN | −2.3418923 | −2.4018146 | 0.04315388 |
| PDSS2 | −2.3382485 | −2.4761292 | 0.03843727 |
| LIG1 | −2.313584528 | −2.906708453 | 0.019758362 |
| BHLHE41 | −2.3051891 | −3.0280385 | 0.01642191 |
| GPT2 | −2.3016776 | −2.3781416 | 0.04477567 |
| PAN2 | −2.2936077 | −2.4550138 | 0.03972186 |
| CDC42BPG | −2.2843117 | −3.5495143 | 0.00755549 |
| ENGASE | −2.2763629 | −2.5253957 | 0.0356012 |
| CSAD | −2.2681778 | −2.3544315 | 0.04646139 |
| MDK | −2.2129106 | −3.7453447 | 0.00569593 |
| NARF | −2.1946011 | −2.3075802 | 0.04998168 |
| PDE4DIP | −2.1444803 | −2.5412632 | 0.03473373 |
| KIAA0415 | −2.105623 | −2.7306888 | 0.02589836 |
| ACOT11 | −2.0705131 | −2.4627636 | 0.03924542 |
| ASCC2 | −2.0330235 | −2.7730476 | 0.0242603 |
| PLEKHG6 | −1.9822023 | −2.8803036 | 0.02057361 |
| AARS2 | −1.977568 | −2.6012709 | 0.03164304 |
| NEDD9 | −1.9619752 | −2.6186277 | 0.03080246 |
| UNC13B | −1.9384901 | −2.6544443 | 0.02913928 |
| GGA1 | −1.922871 | −2.672079 | 0.02835445 |
| ITGB7 | −1.9086556 | −2.7252562 | 0.02611653 |
| HOOK2 | −1.90261 | −2.4030036 | 0.043074 |
| OTUD7B | −1.896354 | −2.4762761 | 0.03842848 |
| CHAF1A | −1.8744616 | −2.4279561 | 0.04143158 |
| RNF115 | −1.8543085 | −3.1780811 | 0.0130914 |
| EZH2 | −1.8459584 | −2.3901176 | 0.0439477 |
| RECQL5 | −1.8306669 | −2.5638143 | 0.0335377 |
| DGKZ | −1.8286593 | −2.3573782 | 0.04624847 |
| AKAP1 | −1.8037181 | −2.7860998 | 0.02377737 |
| GPRC5A | −1.8024252 | −2.3421226 | 0.04736147 |
| DHX8 | −1.7937698 | −2.58587 | 0.03240849 |
| DDB2 | −1.762351611 | −2.659926863 | 0.028892928 |
| RP11-345P4.4 | −1.7574167 | −2.8758358 | 0.02071497 |
| AFMID | −1.7380273 | −2.3643025 | 0.04575199 |
| XPNPEP3 | −1.735687 | −2.3883714 | 0.04406746 |
| C6orf134 | −1.7308082 | −2.340904 | 0.04745152 |
| CEACAM1 | −1.726409623 | −2.531454922 | 0.035267373 |
| GRB7 | −1.720623113 | −2.508716287 | 0.03653679 |
| SIPA1L3 | −1.6693999 | −2.7452524 | 0.02532269 |
| TNFAIP2 | −1.6610635 | −2.4846474 | 0.03793103 |
| MORC2 | −1.6287528 | −2.6187694 | 0.0307957 |
| KIAA0182 | −1.6229045 | −2.9132913 | 0.01956039 |
| TMEM201 | −1.5942392 | −2.6195107 | 0.03076031 |
| KLHL22 | −1.5803749 | −2.5392794 | 0.03484099 |
| LONP1 | −1.5635952 | −2.9330956 | 0.01897711 |
| AKR1C3 | −1.5634478 | −2.5956604 | 0.03191974 |
| DOPEY2 | −1.5025616 | −2.3489132 | 0.04686277 |
| POFUT2 | −1.4878157 | −2.7877209 | 0.02371808 |
| HDAC5 | −1.4512463 | −2.4414893 | 0.04056735 |
| DOT1L | −1.4184125 | −2.5901246 | 0.03219516 |
| CARD14 | −1.4097302 | −2.5029379 | 0.0368667 |
| SS18L1 | −1.3954611 | −2.5587631 | 0.0338019 |
| ACOX3 | −1.3704971 | −2.421331 | 0.0418614 |
| LSG1 | −1.3535336 | −2.3828051 | 0.04445141 |
| CBLC | −1.3526678 | −2.4040419 | 0.04300437 |
| RAD51L3 | −1.2982982 | −2.4528022 | 0.03985889 |
| ACACA | −1.2754988 | −2.4459177 | 0.04028852 |
| USP36 | −1.2523162 | −2.4919107 | 0.0375047 |
| COPB2 | −1.1742598 | −2.318071 | 0.04917093 |

TABLE 3
Genes differentially expressed in progressive
bladder cancers as compared to non-progressive
bladder cancers with a p-value less than 0.01

| SYMBOL | logFC(P/NP) | t | P. Value |
|---|---|---|---|
| IGFBP5 | 5.28378713 | 6.394460723 | 0.000213397 |
| LSP1 | 4.17187565 | 5.47618297 | 0.00059692 |
| STIM1 | 3.42021253 | 4.6546381 | 0.00164936 |
| APOL4 | −3.2213341 | −4.6396991 | 0.00168164 |
| CCPG1 | 2.17244809 | 4.39155853 | 0.00233137 |
| ANTXR2 | 2.25454352 | 4.232024 | 0.00289011 |
| C10orf76 | −3.8186756 | −4.2146327 | 0.00295927 |
| ABCA5 | −2.3816459 | −4.0291014 | 0.003819 |
| TBC1D4 | 2.7293094 | 3.93057252 | 0.00438191 |
| OPTN | 2.1810596 | 3.8899461 | 0.00463941 |
| CYP4Z2P | −3.0769814 | −3.8493161 | 0.00491323 |
| VEGF | −2.408409244 | −3.748738239 | 0.00566838 |
| MDK | −2.2129106 | −3.7453447 | 0.00569593 |
| CYP4B1 | −3.6225716 | −3.7176789 | 0.00592596 |
| AGPS | 2.69481996 | 3.68793759 | 0.00618436 |
| NLRP1 | 3.72742532 | 3.64172336 | 0.00661003 |
| NFIA | 1.83804614 | 3.60419817 | 0.00697863 |
| CDC42BPG | −2.2843117 | −3.5495143 | 0.00755549 |
| SH3D19 | 1.91148603 | 3.54823504 | 0.00756958 |
| KALRN | −3.7281337 | −3.5440781 | 0.00761555 |
| ITGA2 | 2.80098403 | 3.51115212 | 0.00799032 |
| PLA2G2F | −4.156008 | −3.5080194 | 0.00802698 |
| ALDH16A1 | −2.8626969 | −3.5054908 | 0.00805671 |
| LTBP1 | 2.65654311 | 3.4787794 | 0.00837794 |
| DHRS7 | 2.27969466 | 3.46232681 | 0.00858254 |
| 5S_rRNA | 5.02822492 | 3.43620067 | 0.00891841 |
| CASP8 | 2.615453795 | 3.415985067 | 0.009187841 |
| PDLIM5 | 1.70827373 | 3.39620038 | 0.00945989 |
| TRAF4 | −2.350257868 | −3.381925006 | 0.00966147 |
| RAB31 | 2.35711979 | 3.37006139 | 0.00983245 |

The gene names listed in Tables 1, 2 and 3 are common names. NCBI Gene ID numbers for each of the genes listed in Tables 1, 2 and 3 can be obtained by searching the “Gene” Database of the NCBI (available on the World Wide Web at http://www.ncbi.nlm nih.gov/) using the common name as the query and selecting the first returned Homo sapiens gene.

In some embodiments, the methods and assays described herein include (a) transforming the gene expression product into a detectable gene target; (b) measuring the amount of the detectable gene target; and (c) comparing the amount of the detectable gene target to an amount of a reference, wherein if the amount of the detectable gene target is statistically significantly different than the amount of the reference level, the subject is identified as likely to benefit from and/or is administered cystectomy. In some embodiments, if the amount of the detectable gene target is not statistically significantly different than the amount of the reference level, the subject is identified as unlikely to benefit from and/or is not administered cystectomy.

In some embodiments, the reference can be a level of expression of the marker gene product in a population of subjects who have been demonstrated to not benefit from cystectomy. In some embodiments, the reference can be a level of expression of the marker gene product in a population of subjects who have been demonstrated to not be in need of cystectomy. In some embodiments, the reference can be a level of expression of the marker gene product in a population of subjects who have been demonstrated to have non-progressive bladder cancer, e.g. bladder cancer that does not progress from TA and/or T1 to T2. In some embodiments, the reference can also be a level of expression of the marker gene product in a control sample, a pooled sample of control individuals or a numeric value or range of values based on the same.

In certain embodiments, the marker gene(s) are selected from the genes listed in Table 1 and/or Table 2. In certain embodiments, one or more marker genes are selected from the group the genes listed in Table 3. In certain embodiments, one or more marker genes are selected from the group consisting of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5.

In certain embodiments, the marker gene is one of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least two of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least three of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least four of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least five of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least six of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least seven of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least eight of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least nine of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least ten of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least eleven of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least twelve of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least thirteen of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least fourteen of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least fifteen of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include at least sixteen of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5. In certain embodiments, the marker genes include IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5.

In certain embodiments, the marker gene(s) are selected from the group consisting of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least two of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least three of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least four of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least five of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least six of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least seven of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least eight of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least nine of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5. In certain embodiments, the marker genes include at least ten of IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5SrRNA; CASP8; and PDLIM5.

In subjects who are likely to benefit from cystectomy, the marker genes listed in Table 1 can be upregulated and those in Table 2 can be downregulated, e.g. for marker genes listed in Table 1, if the measured marker gene expression in a subject is higher as compared to a reference level of that marker gene's expression, then the subject is identified as likely to benefit from cystectomy. Likewise, for marker genes listed in Table 2, if the measured marker gene expression in a subject is lower as compared to a reference level of that marker gene's expression, then the subject is identified as likely to benefit from cystectomy. Preferably, once looks at a statistically significant change. However, even if a few genes in a group do not differ from normal, a subject can be identified as likely to benefit from cystectomy and/or chemotherapy if the overall change of the group shows a significant change, preferably a statistically significant change.

The level of a gene expression product of a marker gene in Table 1 which is higher than a reference level of that marker gene by at least about 10% than the reference amount, at least about 20%, at least about 30%, at least about 40%, at least about 50%, at least about 80%, at least about 100%, at least about 200%, at least about 300%, at least about 500% or at least about 1000% or more, is indicative that the subject is likely to benefit from cystectomy and/or chemotherapy and/or that the subject should be administered cystectomy and/or chemotherapy in accordance with the methods described herein.

The level of a gene expression product of a marker gene in Table 2 which is lower than a reference level of that marker gene by at least about 10% than the reference amount, at least about 20%, at least about 30%, at least about 40%, at least about 50%, at least about 80%, at least about 90% or more, is indicative that the subject is likely to benefit from cystectomy and/or chemotherapy and/or that the subject should be administered cystectomy and/or chemotherapy in accordance with the methods described herein.

By way of non-limiting example, Table 6 depicts non-limiting potential combinations of two marker genes that can be used in the methods and assays described herein. All possible combinations of 2 or more of the indicated markers are contemplated herein.

TABLE 6

| | IGFBP5 | CD44 | CCND1 | VEGF | TRAF4 | RAB31 | MDK | SNK14 | ANXA1 |
|---|---|---|---|---|---|---|---|---|---|
| IGFBP5 | | X | X | X | X | X | X | X | X |
| CD44 | X | | X | X | X | X | X | X | X |
| CCND1 | X | X | | X | X | X | X | X | X |
| VEGF | X | X | X | | X | X | X | X | X |
| TRAF4 | X | X | X | X | | X | X | X | X |
| RAB31 | X | X | X | X | X | | X | X | X |
| MDK | X | X | X | X | X | X | | X | X |
| SNK14 | X | X | X | X | X | X | X | | X |
| ANXA1 | X | X | X | X | X | X | X | X | |
| CSPG2 | X | X | X | X | X | X | X | X | X |
| CASP8 | X | X | X | X | X | X | X | X | X |
| BIRC2 | X | X | X | X | X | X | X | X | X |
| PAK1 | X | X | X | X | X | X | X | X | X |
| PLA2G2F | X | X | X | X | X | X | X | X | X |
| PICK1 | X | X | X | X | X | X | X | X | X |
| GATA2 | X | X | X | X | X | X | X | X | X |
| ABCA5 | X | X | X | X | X | X | X | X | X |

| | CSPG2 | CASP8 | BIRC2 | PAK1 | PLAS2G2F | PICK1 | GATA2 | ABCA5 |
|---|---|---|---|---|---|---|---|---|
| IGFBP5 | X | X | X | X | X | X | X | X |
| CD44 | X | X | X | X | X | X | X | X |
| CCND1 | X | X | X | X | X | X | X | X |
| VEGF | X | X | X | X | X | X | X | X |
| TRAF4 | X | X | X | X | X | X | X | X |
| RAB31 | X | X | X | X | X | X | X | X |
| MDK | X | X | X | X | X | X | X | X |
| SNK14 | X | X | X | X | X | X | X | X |
| ANXA1 | X | X | X | X | X | X | X | X |
| CSPG2 | | X | X | X | X | X | X | X |
| CASP8 | X | | X | X | X | X | X | X |
| BIRC2 | X | X | | X | X | X | X | X |
| PAK1 | X | X | X | | X | X | X | X |
| PLA2G2F | X | X | X | X | | X | X | X |
| PICK1 | X | X | X | X | X | | X | X |
| GATA2 | X | X | X | X | X | X | | X |
| ABCA5 | X | X | X | X | X | X | X | |

TABLE 10

| | up or down-regulated in progressive bladder | |
|---|---|---|
| | cancers as compared to non-progressive | |
| Gene name | bladder cancers | P value |
| IGFBP5 | up-regulated | 0.0002 |
| ABCA5 | down-regulated | 0.0038 |
| VEGF | down-regulated | 0.0057 |
| PLA2G2F | down-regulated | 0.008 |
| CASP8 | up-regulated | 0.0092 |
| TRAF4 | down-regulated | 0.0097 |
| RAB31 | up-regulated | 0.0098 |
| PICK1 | down-regulated | 0.0113 |
| SNX14 | up-regulated | 0.0145 |
| CD44 | up-regulated | 0.02 |

In certain embodiments, the marker gene(s) are selected from the genes listed in Table 10. In certain embodiments, one or more marker genes are selected from the group consisting of IGFBP5; CD44; VEGF; TRAF4; RAB31; SNX14; CASP8; PLA2G2F; PICK1; and ABCA5. In certain embodiments, the marker gene is one of IGFBP5; CD44; VEGF; TRAF4; RAB31; SNX14; CASP8; PLA2G2F; PICK1; and ABCA5. In certain embodiments,the marker genes comprise at least one of IGFBP5; CD44; VEGF; TRAF4; RAB31; SNX14; CASP8; PLA2G2F; PICK1; and ABCA5. In certain embodiments, the marker genes comprise at least two of IGFBP5; CD44; VEGF; TRAF4; RAB31; SNX14; CASP8; PLA2G2F; PICK1; and ABCA5. In certain embodiments, the marker genes comprise at least three of IGFBP5; CD44; VEGF; TRAF4; RAB31; SNX14; CASP8; PLA2G2F; PICK1; and ABCA5. In certain embodiments, the marker genes comprise at least four of IGFBP5; CD44; VEGF; TRAF4; RAB31; SNX14; CASP8; PLA2G2F; PICK1; and ABCA5. In certain embodiments, the marker genes comprise at least five of IGFBP5; CD44; VEGF; TRAF4; RAB31; SNX14; CASP8; PLA2G2F; PICK1; and ABCA5. In certain embodiments, the marker genes comprise at least six of IGFBP5; CD44; VEGF; TRAF4; RAB31; SNX14; CASP8; PLA2G2F; PICK1; and ABCA5. In certain embodiments, the marker genes comprise at least seven of IGFBP5; CD44; VEGF; TRAF4; RAB31; SNX14; CASP8; PLA2G2F; PICK1; and ABCA5. In certain embodiments, the marker genes comprise at least eight of IGFBP5; CD44; VEGF; TRAF4; RAB31; SNX14; CASP8; PLA2G2F; PICK1; and ABCA5. In certain embodiments, the marker genes comprise at least nine of IGFBP5; CD44; VEGF; TRAF4; RAB31; SNX14; CASP8; PLA2G2F; PICK1; and ABCA5. In certain embodiments, the marker genes comprise at least IGFBP5; CD44; VEGF; TRAF4; RAB31; SNX14; CASP8; PLA2G2F; PICK1; and ABCA5.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

Methods to measure gene expression products associated with the marker genes described herein are well known to a skilled artisan. Such methods to measure gene expression products, e.g., protein level, include ELISA (enzyme linked immunosorbent assay), western blot, and immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques.

For example, antibodies for the polypeptide expression products of the marker genes described herein are commercially available and can be used for the purposes of the invention to measure protein expression levels, e.g. anti-IGFBP5 (Cat. No. 4255; Abeam; Cambridge, MA). Alternatively, since the amino acid sequences for the marker genes described herein are known and publically available at NCBI website, one of skill in the art can raise their own antibodies against these proteins of interest for the purpose of the invention. The amino acid sequences of the marker genes described herein have been assigned NCBI accession numbers for different species such as human, mouse and rat.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations Immunochemistry is a family of techniques based on the use of an antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (i.e. a marker gene polypeptide as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., tumor, blood, serum or urine) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tumor samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent applications Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In certain embodiments, the gene expression products as described herein can be instead determined by determining the level of messenger RNA (mRNA) expression of genes associated with the marker genes described herein. Such molecules can be isolated, derived, or amplified from a biological sample, such as a tumor biopsy. Detection of mRNA expression is known by persons skilled in the art, and comprise, for example but not limited to, PCR procedures, RT-PCR, Northern blot analysis, differential gene expression, RNA protection assay, microarray analysis, hybridization methods, next-generation sequencing etc. Non-limiting examples of next-generation sequencing technologies can include Ion Torrent, Illumina, SOLiD, 454; Massively Parallel Signature Sequencing solid-phase, reversible dye-terminator sequencing; and DNA nanoball sequencing.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art. The nucleic acid sequences of the marker genes described herein have been assigned NCBI accession numbers for different species such as human, mouse and rat. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the nucleic acid molecule to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments of any of the aspects described herein, the level of expression products of more than one gene can be determined simultaneously (e.g. a multiplex assay) or in parallel. In some embodiments, the level of expression products of no more than 200 other genes is determined In some embodiments, the level of expression products of no more than 100 other genes is determined In some embodiments, the level of expression products of no more than 20 other genes is determined In some embodiments, the level of expression products of no more than 10 other genes is determined

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy. In some embodiments, the test sample can be a urine sample.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, a “chemotherapy” refers to a substance that reduces or decreases the growth, survival, and/or metastasis of of cancer cells. Chemotherapies can include toxins, small molecules, polypeptides, and/or radiation therapies. In some embodiments, chemotherapy can include the use of radiation or radiation therapy. In some embodiments, chemotherapy can include, by way of non-limiting example, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN@ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb™); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

The terms “decrease”, “reduced”, or “reduction”, are all used herein to mean a decrease by a statistically significant amount. In some embodiments, “reduce,” “reduction” or “decrease” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or does not encompass a complete inhibition or reduction as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of bladder cancer. A subject can be female or male.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. ovarian cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.),

Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treatment comprising,
   detecting, in a sample obtained from a subject in need of treatment for bladder cancer, the level of expression products of at least one marker gene selected from Table 1 or Table 2;
   administering cystectomy or chemotherapy to the subject if the level of expression products selected from Table 1 are increased relative to a reference level or the level of expression products selected from Table 2 are decreased relative to a reference level; and
   not administering a cystectomy or other invasive treatment to the subject if the level of expression products selected from Table 1 are not increased relative to a reference level or the level of expression products selected from Table 2 are not decreased relative to a reference level.
2. A method of treatment comprising,
   administering cystectomy or chemotherapy to a subject determined to have a level of expression products selected from Table 1 increased relative to a reference level or a level of expression products selected from Table 2 decreased relative to a reference level; and
   not administering a cystectomy or other invasive treatment to a subject determined to have a level of expression products selected from Table 1 not increased relative to a reference level or a level of expression products selected from Table 2 not decreased relative to a reference level.
3. The method of any of paragraphs 1-2, wherein the one or more marker genes is selected from the genes of Table 3.
4. The method of any of paragraphs 1-3, wherein the one or more marker genes is selected from the group consisting of:
   IGFBP5; CD44; CCND1; VEGF; TRAF4; RAB31; MDK; SNX14; ANXA1; CSPG2;
   CASP8; BIRC2; PAK1; PLA2G2F; PICK1; GATA2; and ABCA5.
5. The method of paragraph 4, wherein the level of the expression products of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5 are determined
6. The method of any of paragraphs 1-5, wherein the expression products are mRNA expression products.
7. The method of any of paragraphs 1-6, wherein the expression products are polypeptide expression products.
8. The method of any of paragraphs 1-7, wherein the subject has TA or T1 bladder cancer.
9. The method of any of paragraphs 1-8, wherein the sample is a tumor cell sample.
10. The method of any of paragraphs 1-9, wherein the sample is a urine sample.
11. The method of any of paragraphs 1-10, wherein the subject is a human.
12. An assay comprising, detecting, in a sample obtained from a subject in need of treatment for blader cancer, the level of expression products of at least one marker gene selected from Table 1 or Table 2;
   wherein the subject is likely to benefit from cystectomy or chemotherapy if the level of expression products selected from Table 1 is increased relative to a reference level or the level of expression products selected from Table 2 is decreased relative to a reference level; and
   wherein the subject is not likely to benefit from a cystectomy or other invasive treatment if the level of expression products selected from Table 1 is not increased relative to a reference level or the level of expression products selected from Table 2 is not decreased relative to a reference level.
13. The assay of paragraph 12, wherein the one or more marker genes is selected from the genes of Table 3.
14. The assay of any of paragraphs 12-13, wherein the one or more marker genes is selected from the group consisting of:
   IGFBP5; CD44; CCND1; VEGF; TRAF4; RAB31; MDK; SNX14; ANXA1; CSPG2;
   CASP8; BIRC2; PAK1; PLA2G2F; PICK1; GATA2; and ABCA5
15. The assay of paragraph 14, wherein the level of the expression products of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5 are determined
16. The assay of any of paragraphs 12-15, wherein the expression products are mRNA expression products.
17. The assay of any of paragraphs 12-16, wherein the expression products are polypeptide expression products.
18. The assay of any of paragraphs 12-17, wherein the level of expression of a marker gene product is determined using an method selected from the group consisting of:
   RT-PCR; quantitative RT-PCR; Northern blot; microarray based expression analysis; Western blot; immunoprecipitation; enzyme-linked immunosorbent assay (ELISA); radioimmunological assay (RIA); sandwich assay;
   fluorescence in situ hybridization (FISH); immunohistological staining;
   radioimmunometric assay; immunofluoresence assay; mass spectroscopy and immunoelectrophoresis assay.
19. The assay of any of paragraphs 12-18, wherein the subject has TA or T1 bladder cancer.
20. The assay of any of paragraphs 12-19, wherein the sample is a tumor cell sample.
21. The assay of any of paragraphs 12-20, wherein the sample is a urine sample.
22. The assay of any of paragraphs 12-21, wherein the subject is a human.
23. A method of determing if a subject is likely to benefit from cystectomy, the method comprising, detecting, in a sample obtained from a subject in need of treatment for bladder cancer, the level of expression products of at least one marker gene selected from Table 1 or Table 2;
   wherein the subject is likely to benefit from cystectomy or chemotherapy if the level of expression products selected from Table 1 is increased relative to a reference level or the level of expression products selected from Table 2 is decreased relative to a reference level; and
   wherein the subject is not likely to benefit from a cystectomy or other invasive treatment if the level of expression products selected from Table 1 is not increased relative to a reference level or the level of expression products selected from Table 2 is not decreased relative to a reference level.
24. The method of paragraph 23, wherein the one or more marker genes is selected from the genes of Table 3.
25. The method of any of paragraphs 23-24, wherein the one or more marker genes is selected from the group consisting of:
   IGFBP5; CD44; CCND1; VEGF; TRAF4; RAB31; MDK; SNX14; ANXA1; CSPG2;
   CASP8; BIRC2; PAK1; PLA2G2F; PICK1; GATA2; and ABCA5
26. The method of paragraph 23-25, wherein the level of the expression products of IGFBP5; CD44; CCND1; VEGF; TRAF4; RAB31; MDK; SNX14; ANXA1; CSPG2; CASP8; BIRC2; PAK1; PLA2G2F; PICK1; GATA2; and ABCA5 are determined
27. The method of any of paragraphs 23-26, wherein the expression products are mRNA expression products.
28. The method of any of paragraphs 23-27, wherein the expression products are polypeptide expression products.
29. The method of any of paragraphs 23-28, wherein the level of expression of a marker gene product is determined using an method selected from the group consisting of:
   RT-PCR; quantitative RT-PCR; Northern blot; microarray based expression analysis; Western blot; immunoprecipitation; enzyme-linked immunosorbent assay (ELISA); radioimmunological assay (RIA); sandwich assay;
   fluorescence in situ hybridization (FISH); immunohistological staining;
   radioimmunometric assay; immunofluoresence assay; mass spectroscopy and immunoelectrophoresis assay.
30. The method of any of paragraphs 23-29, wherein the subject has TA or T1 bladder cancer.
31. The method of any of paragraphs 23-30, wherein the sample is a tumor cell sample.
32. The method of any of paragraphs 23-31, wherein the sample is a urine sample.
33. The method of any of paragraphs 23-32, wherein the subject is a human.
34. The method or assay of any of paragraphs 1-33, wherein the marker gene(s) are selected from the group consisting of:
IFGBP5; LSP1; STIM1; APOL4; CCPG1; ANTXR2; C10orf76; ABCA5; TBC1D4; OPTN; CYP4Z2P; VEGF; MDK; CYP4B1; AGPS; NLRP1; NFIA; CDC42BPG; SH3D19; KALRN; ITGA2; PLA2G2F; ALDH16A1; LTBP1; DHRS7; 5S_rRNA; CASP8; and PDLIM5.
35. The method or assay of any of paragraphs 1-34, wherein the one or more marker genes is selected from the group consisting of:
   IGFBP5; CD44; VEGF; TRAF4; RAB31; SNX14; CASP8; PLA2G2F; PICK1; and
   ABCA5.
36. The method or assay of paragraph 35, wherein the level of the expression products of IGFBP5; CD44; VEGF; TRAF4; RAB31; SNX14; CASP8; PLA2G2F; PICK1; and ABCA5 are determined

### EXAMPLES

### Example 1

### Differentiating Progressive from Non-Progressive T1 Bladder Cancer by Gene Expression Profiling: Applying RNA-Seq Analysis on Archived Specimens

Described herein is the identification of gene signatures in transitional cell carcinoma that can differentiate high-grade T1 non-progressive (T1NP) bladder cancer (BCa) from those T1 progressive (T1P) tumors that progress to muscularis propria invasive T2 Tumors.

A high-throughput RNA sequencing (RNA-Seq) was performed on formalin-fixed and paraffin-embedded (FFPE) BCa specimens with clinical pathological characteristics best representing the general clinical development of the disease. For the T1NP group, only patients with long term follow-up (6-17 years) and periodic examinations (average of 4 resections and 9 cytology tests) were selected. For the T1P group, only patients in whom a complete resection was performed after a minimum of at least 8 months after the initial T1 diagnosis were selected, therefore eliminating the possibility of under diagnosis. Only samples in which muscularis propria was present and uninvolved were included, further assuring a correct diagnosis. The RNA-Seq reads were mapped to the human genome build NCBI 36 (hg18) using Tophat with no mismatch. After alignment to the transcriptome and expression quantification, a linear statistical model was built using Limma between T1NP and T1P samples to identify differentially expressed genes.

A total of 5,561 genes were mapped to all samples and used for RNA-Seq analysis to identify a gene signature that was significantly and differentially expressed between T1NP and T1P BCa patients. Signature-based stratification indicated the gene signature correlated notably with the time of T1 development to T2 tumor, suggesting that the molecular signature might be used as an independent predictor for the pace of high-grade T1 BCa progression.

This is the first demonstration that RNA-Seq can be applied as a powerful tool to study BCa using FFPE specimens. A gene signature was identified that can distinguish patients diagnosed with high-grade T1 BCa which remain as non-muscle invasive tumors from those patients with cancers progressing to muscle invasive tumors. These findings will permit prognostic tools for accurate prediction of T1 BCa progression that can considerably influence the clinical decision—making process, treatment regimen and patient survival.

Introduction

Management of high-grade T1 (lamina propria invasive) bladder cancer (BCa) poses a challenging dilemma to clinicians and patients. Most BCa patients present initially as non-muscle invasive transitional cell carcinoma (TCC). There are a significant number of high-grade T1 lesions which have the potential to progress to muscle invasive BCa with increased risk for developing metastatic disease (1-2). For high-grade T1 TCC, approximately 80% of treated tumors recur and 5 to 15%% of recurrent tumors progress to invasive disease. Once BCa becomes metastatic, the cancer related survival is approximately 5.4% at 5 years (3). Currently, no definitive risk criteria exist to distinguish patients with T1 non-progressive (T1NP) BCa who may suffer multiple recurrences of the disease without developing muscle invasive tumors from those patients with the T1 progressive (T1P) BCa whose cancer first presented as T1 disease but eventually developed into muscle invasive and metastatic disease. Therefore, establishing new prognostic criteria to distinguish high-grade T1NP from T1P would meet a great clinical need.

Previously, BCa prognostic factors have been reported and specific markers and molecular pathways have been linked with BCa tumor stage and progression (4-6). Thus far, no standard guidelines have been adopted into clinical practice. Most of these studies used samples pooled from distinct groups of non-invasive BCa (Ta and T1) tumors and compared them with muscularis propria invasive BCa (T2, T3 and T4) tumors. Specimens with long-term clinical T1 BCa follow-up were lacking. This makes distinguishing BCa patients with T1NP from those of T1P particularly difficult, if not impossible from these studies.

Gene expression profiling has been used to develop prognostic signatures in a wide range of diseases (7-8), but its application has been limited, to an extent, by the fact that gene expression technologies work best with fresh frozen tissue (9-11). By far, the vast majority of human disease tissue samples and those with the best outcome data are archived formalin-fixed paraffin-embedded (FFPE) surgical specimens. Important clinical information and disease outcome are often collected years after the initial specimen collection. However, FFPE tissue processing is known to cause fragmentation and chemical modification of RNA, presenting challenges for gene expression profiling. The analysis of BCa specimens is potentially more difficult as they are typically obtained by transurethral resection (TUR) and are associated with cautery artifacts that may further degrade nucleic acids.

High-throughput RNA sequencing (RNA-Seq) is a recently developed gene expression profiling method that has several advantages over other expression profiling technologies, including higher sensitivities, low background noise and the ability to detect splicing variants and somatic mutations, resulting in precise measurements of levels of transcripts and their isoforms (12-13). To this date, the RNA-Seq technology using FFPE samples on BCa analysis has not been published.

The goal of the work described herein was to determine whether RNA-Seq analysis with archival FFPE BCa specimens could be used to identify a genomic signature capable of differentiating high-grade BCa of T1NP from those T1 diseases, which eventually progress to muscle invasive tumors. FFPE samples of BCa patients with long follow-up were used to determine the natural history of the disease. RNA-Seq analysis performed on these FFPE samples identified a gene expression signature associated with disease progression. The results described herein demonstrate the applicability of using RNA-Seq to study bladder tumors obtained by TUR and stored long-term as FFPE specimens. The gene signature identified by this study permit a diagnostic tool for patients at high risk for rapid progression to muscle invasive BCa.

Materials and Methods

Patients and tumor specimens. Research was performed with the approval by the hospital Institutional Review Board (IRB) at Massachusetts General Hospital (MGH). All the samples were obtained from patients treated at MGH between 1994 and 2011. BCa tumor FFPE specimens from 3 T1NP and 4 T1P patients were analyzed for each experiment condition. Specimens of papillary BCa were carefully evaluated by an expert urologic pathologist (CLW) and the tumor tissues were identified, marked and highlighted. Two cores with only papillary bladder tumors and without any stromal or muscular tissues were collected from highlighted areas using a biopsy punch with a plunger (1.5 mm in diameter) in a RNA-free environment. Cancer grade was assigned using the 1973 World Health Organization pathology (WHO) criteria for BCa and cancer stage assigned according to the 7^{th} edition of AJCC TNM system (17-18).

RNA extraction, rRNA removal and sequencing library construction. Total RNA was extracted using hot phenol with additional purification using the RNEASY™ mini kit (Qiagen, Germantown, MD) following the manufacturer's instructions. RNA integrity was assessed using an Agilent bioanalyzer (Agilent, Santa Clara, Calif.) and the RNA integrity number (RIN) was calculated for each sample and the average RIN for all samples was 2.6, ranging from 2.3 to 3.3). A cDNA library was constructed for each sample using Illumina's mRNA-Seq Sample Prep Kit™ (Illumina San Diego, Calif.). Briefly, for each sample, 100 ng of total RNA was used to generate a sequencing library and the RNA was directly subjected to fragmentation without the mRNA purification step. The resulting sample libraries were subjected to DSN (double-specific nuclease) treatment using the Trimmer-Direct cDNA Normalization™ kit (Evrogen, Moscow, Russia).

RNA-Seq read mapping and annotation. The cDNA library of each sample was loaded to a single lane of Illumina flow cell and the libraries were sequenced on Illumina Genome Analyzer II™. Image deconvolution and calculation of quality value were performed using the Boat module (Firecrest v1.1.4.0 and Bustard v.1.4.0 programs) of Illumina pipeline V1.4™. Sequence base calls were assigned using Illumina CASAVA™ software. The reads were 36 bases long and each lane produced an average 30 million of 36-mer raw sequence reads. Reads were mapped to the human genome build NCBI 36 (hg18) using Tophat™ (19) with no mismatch. The mapped reads were assembled and annotated using Cufflinks™ software tools (20).

Transcript quantification and gene expression consolidation. Transcript abundances were quantified in FPKM (Fragments Per Kilobase of exon per Million fragments mapped) by Cufflinks™ which taking into account both the gene length and the mapped reads for each sample and normalized accordingly (21). Due to the fragmented nature of mRNA in FFPE samples, the abundance measurement at the gene level was focused upon. When multiple transcript abundance measurements were reported for a gene, the maximum value was chosen to represent the expression level of that gene.

Differentially expressed genes identification in RNA-Seq data. After alignment to the transcriptome and expression quantification, a linear statistical model was built using limma (21-22) between 3 T1NP samples and 4 T1P samples to identify differentially expressed genes with T1NP samples as the reference. The analyses were accomplished using R™ and Bioconductor™ packages (23). To validate our gene signature, Illumina DASL Cancer Panel™ (The cDNA-mediated Annealing, Selection, extension, and Ligation) Assay was performed independently with the same specimens and genes were identified to show significantly differential expressions between the T1NP and T1P patients using both the RNA-Seq™ and the DASL™ platforms.

Functional enrichment and network analysis. Network enrichment for the significantly differentially expressed genes was analyzed using Ingenuity Pathway Analysis (IPA)™ software. The network interaction of the focused genes in the network is based on their connectivity in Ingenuity Knowledge Base™.

Results

Patient cohort characteristics. To ensure the high specificity of the gene signature obtained, tissue samples were selected from patients with the clinicalpathologial characteristics that best represented the general clinical development of non-progressive from progressive disease. 7 patients in this study were selected based on several considerations. First, all patients had well-established lamina propria invasive TCC confirmed by an expert urologic pathologist. Second, every patient's disease history and their pathology reports were carefully evaluated to ascertain BCa progression. Third, only patients with extended follow-up and sufficient tumor cells in the paraffin blocks were included in this study. All specimens had muscularis propria present which was uninvolved. Patients had a short interval follow-up biopsy after initial diagnosis which demonstrated no tumor. As shown in Table 4, for the T1NP group, these 3 patients were confirmed as non-progressive disease by subsequent biopsies, and with significantly extended follow-up times, ranging from 6 to 17 years (average 9 years). For T1P patients, we excluded any cases with short interval between T1 and T2 diagnosis, eliminating the possibility of T1 BCa being under-staged. The 4 T1P patients had an average time to development of muscularis propria invasive cancer of 2.43 years, ranging from 0.8 to 4.5 years and with an average follow-up time of 4.75 years ranging from 1 to 8 years. All these data coincided with the general clinical observation of T1 BCa progression.

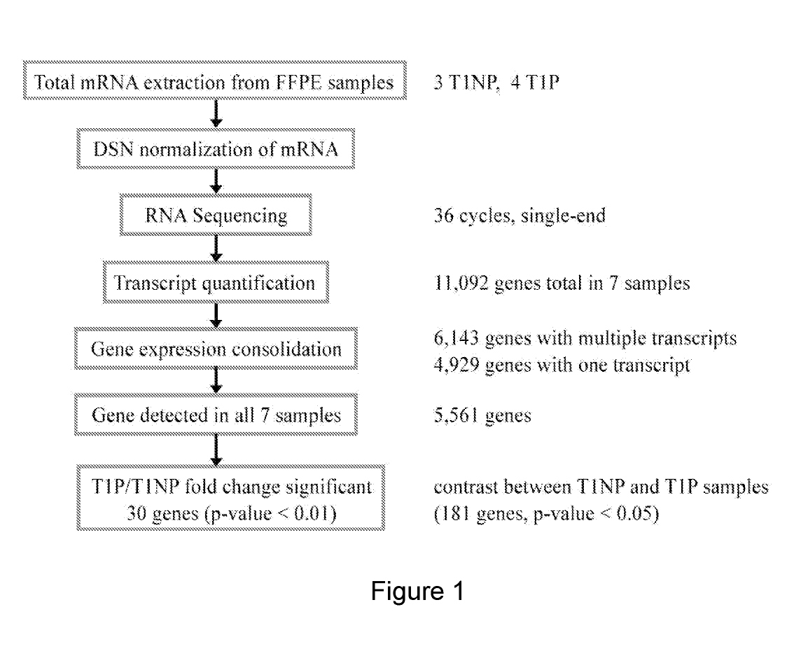

RNA-Seq analysis of T1NP and T1P tumors. RNA-Seg™ analysis was performed using the Illumina GAII™ platform. The study design and the workflow for the RNA-Seq are illustrated in FIG. 1 and are described in detail above herein. After transcript quantification, a total of 11,092 genes were detected in at least one out of the 7 samples with 6,143 genes with multiple transcripts and 4,929 genes with one transcript. An unbiased analysis of the expression data revealed that 5,561 genes were found to be expressed in all samples and it is this final set that were used for further analysis. The characteristics of the RNA-Seq data are summarized in Table 5. On average, about 21 million sequencing reads were generated covering 47 million exon bases. Not all the genes could be mapped to the transcribed database, likely due to genetic variations and repetitive elements and tandem repeats (24). The average number of genes mapped was 8,149, which is substantially smaller then what has been reported when using RNA derived from fresh or frozen samples (25).

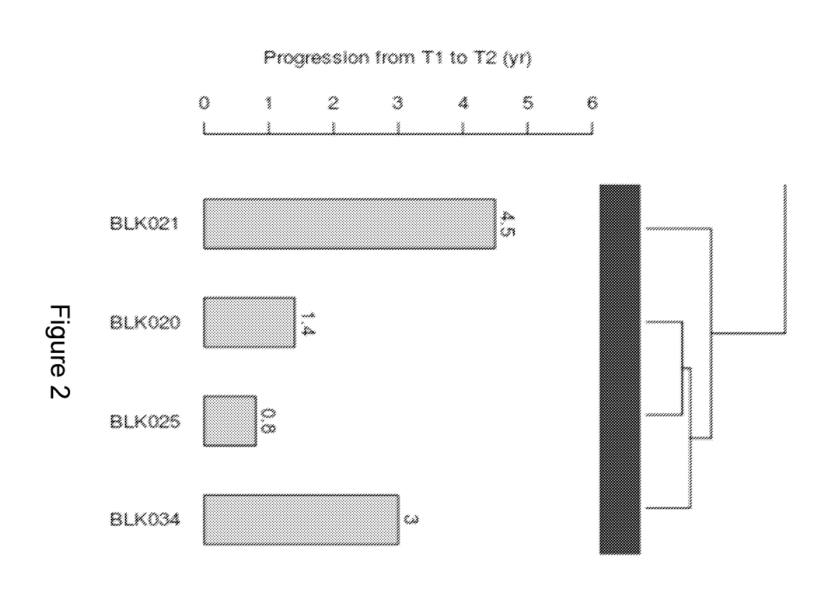

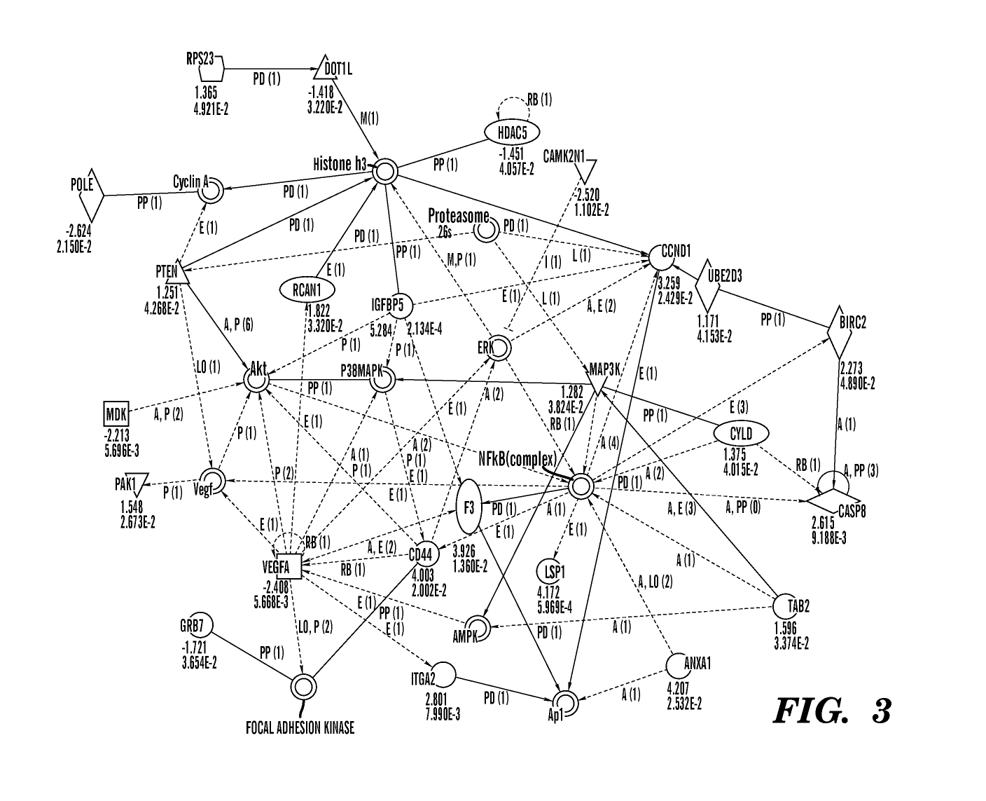

Identification of differentially expressed genes in T1NP compared to T1P samples. Limma™ (22) was used to construct a linear model and identified a total of 181 significantly differentially expressed genes (Tables 1 and 2, p-value <0.05) between the non-progressive and progressive patients whose original diagnosis was high-grade T1 BCa. The 181 most significantly differentially expressed genes (p-value <0.05) that can distinguish high-grade T1NP tumors with non-progressive recurrence from those T1P muscle invasion tumors are listed. The genes were sorted based on the p Value and their expression levels were indicated by the log FC (P/NP) values, genes that over-expressed were with positive logFC (P/NP) values and genes that under-expressed were with negative logFC (P/NP) values. Among them, 101 up-regulated and 80 down-regulated genes in T1P relative to T1NP were found. The results were validated with the DASL Cancer Panel™ analysis using the same samples. Average-linkage hierarchical clustering was then performed using a Pearson correlation-coefficient distance metric using the gene signature identified (data not shown) in 3 T1NP samples and 4 T1P samples and median-centered log₂ (FPKM) for each gene. In examining the resulting hierarchical clustering dendrogram, one can see a correlation between gene expression levels and the time for disease progression for the 4 T1P samples. As shown in FIG. 2, it took 0.8, 1.4, 3 and 4.5 years for patient BLK25, BLK20, BLK 34 and BLK21 respectively, for the disease to progress from T1 to muscle invasive BCa. This suggests that the biomarker identified can also help to predict the pace of progression from high-grade T1 BCa to muscle invasive BCa. In order to further understand the biology underlying the patterns of differential expression, Ingenuity Pathway Analysis (IPA)™ software was used to search for over-representation of biological pathways and annotated gene functional classes among the genes found to be significantly differentially expression between TT1P and T1NP patients and the top enriched network associated with T1 BCa progression was shown in FIG. 3.

Discussion

Genomics has created an unprecedented opportunity to survey expression patterns across the genome and to use the resulting data to develop diagnostic and prognostic biomarkers. However, doing this requires the availability of well-annotated clinical samples with extensive clinical data so that patterns of gene expression can be linked to outcome or other relevant endpoints.

While there are many archival pathological samples reserved as FFPE tissues, these have proven difficult to analyze using most of the available genomic technologies. This is largely due to the fact that the process of creating FFPE samples is known to introduce chemical modification and cross-linking between DNA, RNA and proteins in these samples. In BCa, the situation has proven particularly difficult as specimens are typically obtained through TUR and the cautery effect associated with the procedure may further degrade nucleic acids. To overcome these limitations, a variation on RNA-Seq technology that relies on short nucleic acid fragments coupled with DSN normalization was used. DSN removes ribosomal RNA and other abundant double-stranded DNA and DNA-RNA hybrid complexes, allowing creation of RNA-Seq libraries from most highly degraded samples (26-27). Using this approach, a gene signature was identified that significantly differentially expressed between the non-progressive and progressive patients T1 BCa (Tables 1 and 2). The data described herein demonstrate that FFPE specimens obtained by TUR can be used in RNA-Seq, genome-wide expression analysis.

In the work described herein samples were selected that had an appropriate diagnosis and were of high quality. It is known that up to 25% of T1 G3 tumors are incorrectly staged on initial surgical resection (TURB). Studies without systematically repeated TURB could have been hampered by the lack of patients with correctly documented T1 who subsequently progress at a latter date. It is therefore critical that a re-resection be performed, particularly if muscularis propria is not present in the specimen. In this study, a complete resection was performed for each and all patients with a minimum of at least 8 months after the initial T1 diagnosis. Only samples in which muscularis propria was present and uninvolved were included, further assuring a correct diagnosis. For each patient included in the present study, there were longitudinal data, including TURB and cytology tests (between 14 and 24, ranging from 2-40), followed the original diagnosis (data not shown). For the non-progressive group, one of the most critical issues is the length of the follow-up. In the present study, T1NP patients were followed up to 17 years with periodic examinations (average being more than 4 resections and 9 cytology tests), providing strong evidence to support the assignment of non-progressive status. For the progressive group, the important issue was that a second resection should be performed following an initial T1 diagnosis without evidence of T2 disease. In the present study, a sample was required to have a second resection to be accepted as T1 progression, therefore eliminating the possibility of under diagnosis. Only with careful exclusion of patients not fitting these criteria can one be certain of a true T1 diagnosis and not a misdiagnosed T2 BCa.

Previous studies of superficial papillary BCa have associated specific genomic alterations with the disease, including mutations in and dysregulation of FGFR3, PI3K, KRAS, HRAS, TP53, P16, TSC1, and PTEN, loss of chromosome 9, 9p or 9q as well as loss of RB1 (9-10, 28-29). Recently, mutations in genes responsible for chromatin remodeling were identified (30) and high-throughput assays including RNA-Seq performed on fresh tissue samples have shown promising results, demonstrating molecular assessment can be used for the development of mutation and pathway-based trials of targeted therapy for cancer patients (16). Ingenuity Pathway Analysis (IPA) software was used to search for over-representation of biological pathways and annotated gene functional classes among the genes found to be significant. Among the highest ranking were cancer-related pathways associated with cell death, cellular growth and proliferation, and cell cycle (FIG. 3). The portion of BCa tissue that was primarily epithelial cells was used herein, and any stromal contaminant was limited. Even so, it is conceivable that a small amount of stromal contamination could have occurred, but in view of the small volume, it is unlikely that it influenced the results.

As described herein, a gene signature was identified that was able to distinguish T1NP from T1P patients. The gene signature can distinguish patients diagnosed with high-grade T1 BCa that remain as non-muscle invasive tumors from those patients with cancers progressing to muscle invasive tumors. This is the first demonstration that RNA-Seq can be applied as a tool to study BCa using FFPE specimens. These findings permit improvied clinical T1 BCa treatment regimens and thereby impact patient survival.

### REFERENCES

1. Cookson M S, Hen H W, Zhang Z F et al. The natural history of high risk superficial bladder cancer: 15 year outcome. J Urol 1997; 158:62-67.
2. William S G and Stein J P. Molecular pathways in bladder cancer. Urol Res 2004; 32:373-385.
3. http://seer.cancer.gov/statfacts/html/urinb.html#survival
4. Sanchez-Carbayo M, Socci N D, Lozano J, Saint F, Cordon-Cardo C. Defining molecular profiles of poor outcome in patients with invasive bladder cancer using oligonucleotide microarrays. J Clin Oncol 2006; 24:778-89.
5.Schiffer E, Vlahou A, Petrolekas A, Stravodimos K, Tauber R, Geschwend J E, et al. Prediction of muscle-invasive bladder cancer using urinary proteomics. Clin Cancer Res. 2009; 15:4935-4943.
6.Wiklund E D, Bramsen J B, HuIf T, Dyrskjφt L, Ramanathan R, Hansen T B, et al. Coordinated epigenetic repression of the miR-200 family and miR-205 in invasive bladder cancer. Int J Cancer 2011 128:1327-1334.
7. Golub T R, Slonim D K, Tamayo P, Huard C, Gaasenbeek M, Mesirov J P, et al. Molecular classification of cancer: class discovery and class prediction by gene expression monitoring. Science 1999; 286:531-537.
8.Stuart R O, Wachsman W, Berry C C, et al. In silico dissection of cell-type-associated patterns of gene expression in prostate cancer. Proc Natl Acad Sci USA 2004; 101:615-20.
9. Blaveri E, Brewer J L, Roydasgupta R, et al. Bladder cancer stage and outcome by array-based comparative genomic hybridization. Clin Cancer Res 2005; 11:7012-22.
10. Blaveri E, Simko J P, Korkola J E, et al. Bladder cancer outcome and subtype classification by gene expression. Clin Cancer Res 2005; 11:4044-4055.
11. Dyrskjot L, Kruhoffer M, Thykjaer T, et al. Gene expression in the urinary bladder: a common carcinoma in situ gene expression signature exists disregarding histopathological classification. Cancer Res 2004; 64:4040-4048.
12. Mardis E R. The impact of next-generation sequencing technology on genetics. Trends Genet 2008; 24:133-141.
13. Wang Z, Gerstein M, Snyder M. RNA-Seq: a revolutionary tool for transcriptomics. Nat Rev Genet 2009; 10:57-63.
14. Huang Q, Lin B, Liu H, Ma X, Mo F, Yu W, et al. RNA-Seq analyses generate comprehensive transcriptomic landscape and reveal complex transcript patterns in hepatocellular carcinoma. PLoS One 2011; 6:e26168.
15. Berger M F, Levin J Z, Vijayendran K, Sivachenko A, Adiconis X, et al. Integrative analysis of the melanoma transcriptome. Genome Res 2010; 20: 413-427.
16. Roychowdhury S, Iyer M K, Robinson D R, Lonigro R J, Wu Y M, Cao X, et al. Personalized oncology through integrative high-throughput sequencing: a pilot study. Sci Transl Med 2011; 3:1-10.
17. Mostofi F K, Sobin L H, Torloni H. International histology classification of tumours, No. 10. Geneva: World Organization; 1973 Histological typing of urinary bladder tumours.
18. Edge S B, Byrd D R, Compton C C, Fritz A G, Freene F L, Trotti A, editors. American Joint Committee on Cancer (AJCC) cancer staging manual. 67h ed. New York, N.Y.:Springer-Verlag; 2010.
19. Trapnell, C, Pachter L, Salzberg S L. TopHat: Discovering splice junctions with RNA-Seq. Bioinformatics 2009; 25: 1105-1111.
20. Trapnell C, Williams B A, Pertea G, Mortazavi A M, Kwan G, van Baren M J, et al. Transcript assembly and quantification by RNA-Seq reveals unannotated transcripts and isoform switching during cell differentiation. Nat Biotechnology 2010; 28: 511-515.
21. Mortazavi A, Williams B A, McCue K, Schaeffer L, Wold B. Mapping and quantifying mammalian transcriptomes by RNA-Seq. Nat Methods 2008; 5: 621-628.
22.Smyth G K. Limma: linear models for microarray data. Bioinformatics and Computational Biology Solutions using R and Bioconductor. Gentleman, R, Carey S, Dudoit R and Huber (editors). Spring, New York 2005; 397-420.
23. http://www.bioconductor.org/24.Li
24. B, Ruotti V, Stewart R M, Thomson J A, Dewey C N. RNA-Seq gene expression estimation with read mapping uncertainty. Bioinformatics 2010; 26: 493-500.
25. Toung J M, Morley M, Li M, Cheung V G. RNA-sequence analysis of human B-cells. Genome Research 2011 June; 21(6): 991-8
26. Rupp G M, Locker J. Purification and analysis of RNA from paraffin-embedded tissues. Biotechniques. 1988; 6:56-60.
27. Shujun Luo S, Khrebtukova I, Perou C, Schroth G. P. Complete RNA-seq analysis of cancer transcriptomes from FFPE samples. Genome Biol. 2010; 11: sl-P35.
28. Lindgren D, Frigyesi A, Gudjonsson S, Sjodahl G, Hallden C, Chebil G, et al. Combined gene expression and genomic profiling define two intrinsic molecular subtypes of urothelial carcinoma and gene signatures for molecular grading and outcome. Cancer Res. 2010; 70:3463-3472.
29. McConkey D J, Lee, S, Choi W, Tran M, Majewski t, Lee S et al. Molecular genetics of bladder cancer: Emerging mechanisms of tumor initiation and progression. Uro Oncology 2010 28:429-440.
30. Gui Y, Guo G, Huang Y, Hu X, Tang A, Gao S, et al. Frequent mutations of chromatin remodeling genes in transitional cell carcinoma of the bladder. Nat Genet 2011; 43:875-878.

TABLE 4
Clinical Pathological Characteristics of Study Cohort

| | T1 Non-Progressive | | T1 Progressive | |
|---|---|---|---|---|
| | (T1NP) | | (T1P) | |
| Samples | n = 3 | | n = 4 | |
| Median Age years (range) | 69 | (56-79) | 73 | (62-85) |
| Male:Female | 2:1 | | 3:1 | |
| Clinical Tumor Stage | T1 | | T1 | |
| T1 | 3 | | 4 | |
| Grade 1-2 | 0 | | 0 | |
| Grade 3 | 3 | | 4 | |
| Average Follow-up years (range) | 9 | (6-17) | 4.75 | (1-8) |

| | | | |
|---|---|---|---|
| Average Progression years | 0 | 2.43 | (0.8-4.5) |
| (range) | | | |

(N/A—not applicable)

TABLE 5
RNA-Seq Data Summary. The read coverage of the
3 T1NP and 4 T1P patients' samples are shown.

| | | | | Number of | Number of |
|---|---|---|---|---|---|
| Sample | Block | BC | Number | detected | exon- bases |
| ID | Age (yr) | group | of reads | genes | covered |
| BLK004 | 11 | NP | 21879070 | 8790 | 42922505 |
| BLK007 | 16 | NP | 26555690 | 9092 | 47994767 |
| BLK016 | 10 | NP | 33927499 | 7567 | 43266436 |
| BLK020 | 5 | P | 10888159 | 7358 | 50383266 |
| BLK021 | 15 | P | 25313779 | 8935 | 48868444 |
| BLK025 | 6 | P | 18278586 | 8789 | 35467184 |
| BLK034 | 6 | P | 12497222 | 6513 | 61692969 |

### Example 2

### Differentiating Progressive from Non-Progressive T1 Bladder Cancer by Gene Expression Profiling: Applying RNA-Seq Analysis on Archived Specimens

Management of high-grade T1 (lamina propria invasive) bladder cancer (BCa) poses a challenging dilemma to clinicians and patients. Currently, no definitive prognostic criteria exist to help in treatment decision making for high-grade T1 BCa patients. As described herein, gene expression biomarkers were identified in transitional cell carcinoma that can differentiate high-grade T1 non-progressive BCa from those T1 tumors that eventually progress to T2 (muscularis propria invasive) tumors. High-throughput RNA sequencing (RNA-Seq) was performed on BCa specimens obtained by transurethral resection (TUR), as formalin-fixed and paraffin-embedded (FFPE) with a confirmed history of disease development and extended follow-up. Surgical specimens of three T1 non-progressive and four T1 progressive tumors were used. A total of 5,561 genes were mapped to all samples and used to identify significantly and differentially expressed genes between the non-progressive and the progressive T1 BCa patients (p<0.05). Signature-based stratification indicates that the gene signature correlates notably with the time of T1 development to T2 tumor, suggesting that the molecular signature might be used as an independent predictor for the rate of high-grade T1 BCa progression. The data described herein demonstrate that RNA-Seq could be applied as a powerful tool to study BCa gene expression using FFPE samples and permit improved T1 BCa treatment regimens and thereby impact patient survival.

Introduction

Bladder cancer (BCa) is among the five most commonly diagnosed malignancies in the world and is one of the most prevalent types of cancer, responsible for annual deaths of approximately 150,000 worldwide (1-3). Most BCa patients present initially as non-muscle invasive transitional cell carcinoma (TCC), including non-invasive papillary TCC, TCC in situ and lamina propria invasive TCC (stage T1 cancer). Significant cases of T1 lesions are high-grade T1 lesions and have the potential to progress to muscularis propria or muscle invasive BCa with increased risk for developing metastatic cancer (4, 5). For high grade T1 TCC, approximately 80% of treated tumors recur and 20% of recurrent tumors progress to invasive disease. Once BCa becomes metastatic, despite aggressive treatments such as surgical resection, radiotherapy and chemotherapy, treatment fails in 95% patients with advanced disease and th cancer related survival is approximately 5.4% at 5 years (6). Currently, no definitive risk criteria exist to distinguish patients with T1 non-progressive (T1NP) BCa who may suffer multiple recurrences of the disease without developing muscle invasive tumors from those patients with the T1 progressive (T1P) BCa whose cancer first presented as T1 disease but eventually developed into muscle invasive and/or metastatic tumors. Most experts would not recommend immediate cystectomy to prevent progression, given the morbidity of the procedure and the fact that the majority of patient's with T1 disease do not progress and their tumors will not threaten their lives. Therefore, establishing new prognostic criteria to distinguish high-grade T1NP from T1P would meet a great clinical need.

In the past several years, studies have found distinct genotypic and phenotypic patterns to be associated with different stages and progression of BCa. Although prognostic factors including grade, size, recurrence and multiplicity have been reported and specific markers and molecular pathways have been linked and correlated with BCa tumor stage and progression (7-9), thus far, no standard guidelines have been adopted into clinical practice. Most of these studies used samples pooled from distinct groups of non-invasive BCa (Ta and T1) tumors and compared them with muscularis propria invasive BCa (T2, T3 and T4) tumors and specimens with longterm clinical T1 BCa follow-up were lacking. This makes distinguishing BCa patients with T1NP from those of T1P particularly difficult, if not impossible. The current limited prognostic indicators have failed to adequately discriminate T1 patients with the relatively indolent T1NP BCa from those patients with the aggressive invasive T1P BCa.

Gene expression profiling has been used to develop prognostic signatures in a wide range of diseases (10-11), but its application has been limited, to an extent, by the fact that gene expression technologies work best with fresh frozen tissue (12-14). By far, the vast majority of human disease tissue samples, and those with the best outcome data are archived formalin-fixed paraffin-embedded (FFPE) surgical specimens. Important clinical information and disease outcome are often collected years after the initial specimen collection. However, FFPE tissue processing is known to cause fragmentation and chemical modification of RNA, presenting challenges for gene expression profiling. The analysis of BCa specimens is potentially more difficult as they are typically obtained by transurethral resection (TUR) and are associated with cautery heat artifacts that may further degrade nucleic acid. It is unknown if the time FFPE kept in storage would also pose technical difficulties.

High-throughput RNA sequencing (RNA-Seq) is a recently developed gene expression profiling method that has several advantages over other expression profiling technologies, including higher sensitivities, low background noise and the ability to detect splicing variants and somatic mutations, resulting in precise measurements of levels of transcripts and their isoforms (15-16). To this date, the RNA-Seq technology using FFPE samples on bladder tumor analysis has not been published.

The objective of the work described herein was to determine whether RNA-Seq using archival FFPE BCa specimens could be used to identify a genomic signature capable of differentiating high-grade BCa of T1NP from those T1 diseases, which eventually progress to muscle invasive tumors (T2 or higher stages). FFPE samples of BCa patients with extended years of follow-up to determine the natural history of the disease were used. A total of 7 cases, including 3 high-grade T1NP with follow-up time ranging from 6 to 17 years and 4 high-grade T1P whose disease progressed from T1 to muscularis propria invasive tumor following the initial diagnosis in the period between 0.8 to 4.5 years. RNA-Seq analysis was performed on these FFPE samples and a gene expression signature associated with disease progression was identified. Among the top differentially expressed genes are those involved in cell growth and apoptosis and the gene signature described herein correlated considerably with T1 BCa progression. These results demonstrated the applicability of using RNA-Seq to study bladder tumors obtained by TUR and stored in long-term as FFPE specimens. The gene signature identified by this study represents a promising diagnostic tool for patients at high risk for rapid progression to muscle invasive BCa.

Materials and Methods

Patient and tumor samples. Research was performed with the approval by the hospital Institutional Review Board (IRB) at Massachusetts General Hospital. All the samples were obtained from patients treated at Massachusetts General Hospital, between 1994 and 2011. 7 patients in this study were selected based on several considerations (detailed in the Results section). Tumor specimens from 3 T1 non-progressive BCa and 4 T1 progressive BCa patients were analyzed. Tissues were collected by TUR and stored as FFPE specimens. Cancer specimen slides were reviewed and chosen by an expert urologic pathologist (CLW). Cancer grade was assigned using the 1973 World Health Organization pathology (WHO) criteria for bladder cancer and cancer stage assigned according to the 7th edition of AJCC TNM system (20, 21). Two 1 5 mm punched cores were obtained from the tumor area in the paraffin block and subjected to total RNA extraction.

Histological staining and Images collection. The paraffin-embedded bladder cancer tissues were cut at 5 μm and deparaffinized with xylene and rehydrated, following by hematoxylin and eosin (H&E) histological staining The slides were examined by an expert urologic pathologist and the images of representative regions of T1 bladder cancer were collected.

RNA extraction, rRNA removal and sequencing library construction. Total RNA was extracted using hot phenol with additional purification using the RNeasy mini kit™ (Qiagen, Germantown, MD) following the manufacturer's instruction. The quality and quantity of the extracted RNA were evaluated. RNA integrity was assessed using an Agilent bioanalyzer (Agilent, Santa Clara, Calif.) and the RNA integrity number (RIN) was calculated for each sample.

A cDNA library was constructed for each and all 7 samples using Illumina's mRNA-Seq Sample Prep Kit™ (Illumina San Diego Calif.). Briefly, for each sample, 100 ng of total RNA was used to generate a sequencing library without any additional other treatment. The RNA was directly subjected to fragmentation without the mRNA purification step. The first and the second-strand cDNA were synthesized from the fragmented RNA using random hexamer primers. End repair, A-tailing, adaptor ligation, cDNA template purification and enrichment of the purified cDNA template using PCR were then performed. The resulting sample libraries were subjected to DSN™ (double-specific nuclease) treatment using the Trimmer-Direct cDNA Normalization kit™ (Evrogen, Moscow, Russia).

RNA-Seq read mapping and annotation. The cDNA library of each sample was loaded to a single lane of Illumina™ flow cell and the libraries were sequenced on Illumina Genome Analyzer II™. Image deconvolution and calculation of quality value were performed using the Boat module (Firecrest v1.1.4.0 and Bustard v.1.4.0 programs) of Illumina pipeline Vi.4™. Sequence base calls were assigned using Illumina CASAVA™ software. The reads were 36 bases long and each lane produced an average 30 million of 36-mer raw sequence reads. Reads were mapped to the human genome build NCBI 36™ (hg18) using Tophat™ (22) with no mismatch. The mapped reads were assembled and annotated using Cufflinks™ software tools (23).

Transcript quantification and gene expression consolidation. Transcript abundances were quantified in FPKIVI™ (Fragments Per Kilobase of exon per Million fragments mapped) by Cufflinks™. Due to the fragmented nature of mRNA in FFPE samples, the abundance measurement at the gene level was focused upon. When multiple transcript abundance measurements were reported for a gene, the maximum value was chosen to represent the expression level of that gene.

Differentially expressed genes identification in RNA-Seq data. After alignment to the transcriptome and expression quantification, a linear statistical model was built using limma™ (24-25) between 3 T1NP samples and 4 T1P samples to identify differentially expressed genes with T1NP samples as the reference. The analyses were accomplished using R™ and Bioconductor™ packages (26).

DASL Microarray Assay. The Illumina DASL™ (cDNA-mediated Annealing, Selection, extension and Ligation) Human Cancer Panel™ gene set (Illumina Inc., San Diego, Calif., USA) were represented by a pool of selected probe groups that target 502 gene mRNAs collected from publicly available cancer gene lists, including oncogenes, tumor suppressor genes and genes in their associated pathways.

Each mRNA was targeted in three locations by three separate probes. In this study, following manufacture's instruction, briefly, A 100 ng portion of total RNA from BCa samples was converted into cDNA using biotinylated random nonamers, oligo-deoxythymidine 18 primers and Illumina-supplied reagents, according to manufacturer's instructions. The resulting biotinylated cDNA was annealed to assay oligonucleotides and bound to streptadivin-conjugated paramagnetic particles to select the cDNA/oligo complexes. After oligo hybridization, mis-hybridized and non-hybridized oligos were washed away, while bound oligos were extended and ligated to generate templates to be subsequently amplified with shared PCR primers. For each sample, at least three technical replicates were performed. After hybridization, the arrays were scanned by laser confocal microscopy using the Illumina BeadArrary Reader 500™ system. To identify significantly differentially expressed genes between T1NP and T1P samples in DASL Cancer Panel™ data, the same linear modeling as in RNA-Seq was applied.

Functional enrichment and network analysis. Network enrichment for the significantly differentially expressed genes was analyzed using Ingenuity Pathway Analysis (IPA)™ software. The network interaction of the focused genes in the network is based on their connectivity in Ingenuity Knowledge Base™.

Results

Patients’ FFPE sample selection. To ensure the high specificity of the gene signature obtained, tissue samples for which the clinicalpathologial characteristics that best represent the general clinical development of the disease were selected. 7 patients in this study were selected based on several considerations. First, all patients had well-established lamina propria invasive TCC confirmed by an expert urologic pathologist. Second, every patient's disease history and their pathology reports were carefully evaluated to ascertain BCa progression. Third, only patients with extended follow-up and sufficient tumor cells in the paraffin blocks were included in this study. As shown in Table 4, for the T1NP group, these 3 patients were confirmed as non-progressive disease by subsequent biopsies, and with significantly extended follow-up times, ranging from 6 to 17 years (average 9 years). For T1P patients, it was recognized that some T1 cancers could be under-staged. When selecting the cases for the present study, any cases with very short interval between T1 and T2 diagnosis were excluded. The 4 patients in the T1P group had an average time to development of muscularis propria invasive cancer of 2.43 years, ranging from 0.8 to 4.5 years and with an average follow-up time of 4.75 years ranging from 1-8 years (Table 4). All these data coincided with the general clinical observation of T1 BCa progression. The representative images collected from both T1NP (FIG. 1A) and T1P (FIG. 1B) BCa patient samples were collected (data not shown). Bearing in mind the heterogeneity of the disease and the potential contaminants of the samples, a considerable effort was made to ensure the quality and quantity of the FFPE samples.

RNA-Seq analysis. Specimens of papillary BCa were carefully evaluated by an expert urologic pathologist and the tumor tissues were identified, marked and highlighted. FFPE cores were collected from these areas using a biopsy punch with a plunger (1.5 mm in diameter) in a RNA-free environment and all the excess amount of paraffin was removed. Once the RNA was extracted, the quantity of extracted RNA was examined and sample integrity was further evaluated on an Agilent Bioanalyzer.

RNA-Seq analysis of T1NP and T1P tumors. RNA-Seq was performed using the Illumina GAII™ platform. The study design and the workflow for the RNA-Seq are illustrated in FIG. 2 and are described in details in materials and methods. Briefly, the total RNA from FFPE tissue samples was purified as described in materials and methods. DSN normalization was performed after RNA-Seq sample preparation and before cluster generation. It involved the degradation of abundant cDNA molecules derive from rRNA, tRNA and housekeeping genes while preserving those derived from less abundant transcripts.

As shown in FIG. 1, after transcript quantification, a total of 11,092 genes found to be detected in at least one out of the 7 samples with 6,143 genes with multiple transcripts and 4,929 genes with one transcript. An unbiased analysis of the expression data revealed that 5,561 genes were found to be expressed to all samples and it is this final set that were used for further analysis. There are total of 6869 genes expressed in 3 T1NP samples, which resulted in 1335 genes only expressed in 3 T1NP samples. Conversely, there are total of 5778 genes expressed in 4 T1P samples, which resulted in 217 genes only expressed in 4 T1NP samples. The characteristics of the RNA-Seq data are summarized in Table 5. On average, about 21 million sequencing reads were generated covering 47 million of exon bases. Not all the genes could be mapped to the transcribed database, likely due to genetic variations and repetitive elements and tandem repeats (27). The average number of genes mapped was 8,149, which is substantially smaller then what has been reported when using RNA derived from fresh or frozen samples. In cultured human B cells 20,776 genes were reported by Toung et. al. in their RNA-Seq experiment (28). In fresh frozen sarcoma tissues, 18,835 to 20,515 genes were identified (unpublished data, personal communications). The older specimens had higher number of reads obtained, as shown in Table 5. These data suggest that improvement in FFPE sample preparation and storage may have a direct impact on molecular profiling.

Identification of differential expressed genes in T1NP compared to T1P samples. Limma™ (25) was used to construct a linear model and a total of 181 significantly differentially expressed genes between the non-progressive and progressive groups of patients (p-value <0.05) whose original diagnosis were high-grade T1 BCa (Tables 1 and 2) was identified. Among these, 101 genes were up regulated and 80 were down regulated in T1P relative to T1NP. Table 7 lists the top 28 most significantly differentially expressed genes (p-value <0.01) that can distinguish high-grade T1NP tumors with non-progressive recurrence from those T1P muscle invasion tumors. Average-linkage hierarchical clustering was performed using a Pearson correlation-coefficient distance metric using the gene signature identified (FIG. 2). In examining the hierarchical clustering dendrogram, one can see a correlation between gene expression levels and the time for disease progression for the 4 T1P samples. As shown in FIG. 2, it took 0.8 year for patient BLK25, 1.4 year for patient BLK20, 3 years for patient BLK 34 and 4.5 years for patient BLK21 for the disease to progress from T1 to muscle invasive BCa. This suggests that the biomarker identified herein may also help to predict the pace of progression from high-grade T1 BCa to muscle invasive BCa.

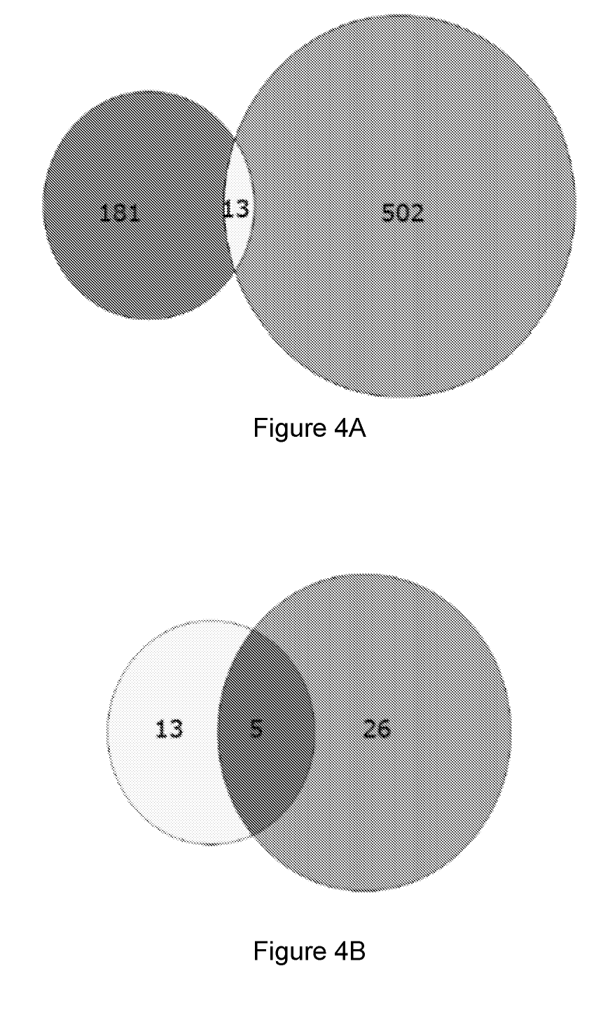

Application of DASL array assay to identify differential expressed genes between T1NP and T1P FFPE samples. The DASL Cancer Panel assay has showed limited but demonstrated ability to profile a significant number of FFPE cancer samples. It has generated gene expression profiles from FFPE samples and other samples containing partially degraded RNAs (29-30). To validate the RNA-Seq result described above, DASL caner panel analysis was performed with the same cohort on all the 7 patients' samples. This method enables the measurement RNA abundance of over 502 genes in parallel per sample. Analysis was performed on the DASL Cancel Panel and 218 genes (among the 502 gene) were expressed in all blocks and 150 genes were not. A Total of 13 genes on the DASL Cancer Panel were present in the presentlyd escribed 181-gene signature identified by RNA-Seq using the same samples to distinguish T1NP from T1P (As shown in FIG. 4A). An independent analysis demonstrated that 5 of the 13 genes showed significantly differential expressions between the T1NP and T1P patients (FIG. 4B) using both the RNA-Seq and the DASL analyses. Furthermore, when the Spearman correlation coefficient (rank of the expression among the samples, not the expression value itself) was calculated between RNA-Seq and DASL data, a high degree of correlation was found (shown in Table 8, VEGF=0.82 CCND1=0.86, CD44=0.93, TRAF4=0.93, IGFBP5=0.82).

The top enriched network associated with BCa progression. Ingenuity Pathway Analysis (IPA) software was used to search for over-representation of biological pathways and annotated gene functional classes among the genes found to be significant. Among the significant genes, 193 significant annotated biological functions (Table 8) and 68 over-represented canonical pathways (Fisher's Exact Test P value <0.05) were found within the expression gene signature (Table 9) with the top enriched network associated with BCa progression shown in FIG. 3. Among the highest ranking were many cancer-related pathways associated with cell death, cellular growth and proliferation, and cell cycle.

Discussion

Genomics has created an unprecedented opportunity to survey expression patterns across the genome and to use the resulting data to develop diagnostic and prognostic biomarkers. However, doing this requires the availability of well-annotated clinical samples with extensive clinical data so that patterns of gene expression can be linked to outcome or other relevant endpoints.

While there are many of archival pathological samples reserved as formalin-fixed paraffin-embedded (FFPE) tissues, these have proven difficult to analyze using most of the available genomic technologies. This is largely due to the fact that the process of creating FFPE samples is known to introduce chemical modification and cross-linking of RNA molecules through the addition of mono-methylol to amino groups for RNA and cross-linking between DNA, RNA and proteins occur in these samples. In BCa, the situation has proven particularly difficult as specimens are typically obtained through TUR and the cautery effect associated with the procedure may further degrade nucleic acid.

To overcome these limitations, a variation on RNA-Seq technology that relies on short nucleic acid fragments coupled with double-stranded nuclease (DSN) normalization was used herein. DSN removes ribosomal RNA and other abundant double-stranded DNA and DNA-RNA hybrid complexes, allowing creation of RNA-Seq libraries from most highly degraded samples (31-32). Using this approach, it is demonstrated herein that FFPE specimens obtained by TUR could be used in RNA-Seq, genome-wide expression analysis. The present data suggest that the length of FFPE specimen storage is not associated with reduced RNA quantity or quality (Table 4).

In this study, DASL Caner Panel analysis was performed on all the 7 samples and 13 genes were present on the gene signature identified by RNA-Seq. Surprisingly, out of the 13 genes on the list, an independent analysis demonstrated that 5 of the 13 genes showed significantly differential expressions between the T1NP and T1P patients when comparing between RNA-Seq and DASL data. Moreover, a high degree of correlation was found (Spearman correlation coefficient range from 0.82 to 0.93). This is extremely encouraging, given the challenge of the FFPE samples and the fundamental differences between the two approaches.

Previous studies of superficial papillary bladder tumors have associated with etiologic factors such and smoking and specific genomic alterations with the disease, including mutations in and dysregulation of FGFR3, PI3K KRAS, HRAS, TPp53, P16, TSC1, and PTEN, loss of chromosome 9, 9p or 9q as well as frequent amplification of 6p22 and loss of RB1 (12, 13, 33-46). To date, studies have not produced clinical useful prognostic predictors for T1 BCa progression. Described herein is the identification of a gene signature that was able to distinguish T1NP from T1P patients.

TABLE 7
Annotation of top differentially expressed genes. The top 28 most significantly differentially expressed genes (p-value < 0.01)
that can distinguish high-grade T1NP tumors with non-progressive recurrence from those T1P muscle invasion tumors was listed.

| Gene Symbol | Gene Name | Function Annotation | P. Value |
|---|---|---|---|
| IGFBP5 | insulin-like growth factor binding protein 5 | regulation of cell growth | 2.13 × 10⁻⁴ |
| LSP1 | lymphocyte-specific protein 1 | apoptosis | 5.97 × 10⁻⁴ |
| STIM1 | stromal interaction molecule 1 | calcium ion binding | 1.65 × 10⁻³ |
| APOL4 | apolipoprotein L, 4 | lipid transport | 1.68 × 10⁻³ |
| CCPG1 | cell cycle progression 1 | cell cycle | 2.33 × 10⁻³ |
| ANTXR2 | anthrax toxin receptor 2 | ion binding, lipid metalolism | 2.89 × 10⁻³ |
| C10orf76 | chromosome 10 open reading frame 76 | region of unkown function DUF1741 | 2.96 × 10⁻³ |
| ABCA5 | ATP-binding cassette, sub-family A (ABC1), member 5 | ABC tmsporters | 3.81 × 10⁻³ |
| TBC1D4 | TBC1 domain family, member 4 | small GTPase regulator activity | 4.38 × 10⁻³ |
| OPTN | oplineurin | ion binding, cell division | 4.64 × 10⁻³ |
| CYP4Z2P | cytochrome P450 family4 subfamily Z, polypeptide 2 pseudogene | oxidation reduction | 4.91 × 10⁻³ |
| VEGF | vascular endothelial growth factor | growth factor activity, cell division | 5.67 × 10⁻³ |
| MDK | midkine | growth factor activity, cell division | 5.69 × 10⁻³ |
| CYP4B1 | cytochrome P450, family 4, subfamily B, polypeptide 1 | secondary metabolites biosythesis | 5.92 × 10⁻³ |
| AGPS | alkylglycerone phosphate synthase | Ether lipid metabolism | 6.18 × 10⁻³ |
| NLRP1 | NLR family, pyrin domain containing 1 | apoptosis | 6.61 × 10⁻³ |
| NFIA | nuclear factor I/A | DNA metabolic process | 6.98 × 10⁻³ |
| CDC42BPG | CDC42 binding protein kinase gamma (DMPK-like) | protein phosphorylation | 7.56 × 10⁻³ |
| SH3D19 | SH3 domain containing 19 | regulation of catabolic process | 7.57 × 10⁻³ |
| KALRN | kalirin, RhoGEF kinase | small GTPase regulator activity | 7.62 × 10⁻³ |
| ITGA2 | integrin, alpha 2 (CD49B, alpha 2 subunit of VLA-2 receptor) | cell adhesion | 7.99 × 10⁻³ |
| PLA2G2F | phospholipase A2, group IIF | carboxylesterase activity | 8.03 × 10⁻³ |
| ALDH16A1 | aldehyde dehydrogenase 16 family, member A1 | oxidation reduction | 8.06 × 10⁻³ |
| LTBP1 | atent transforming growth factor beta binding protein 1 | rotein kinase activity | 8.37 × 10⁻³ |
| DHRS7 | dehydrogenase/reductase (SDR family) member 7 | oxidation reduction | 8.58 × 10⁻³ |
| 5S_rRNA | 5S ribonucleoprotein | Ribosome | 8.92 × 10⁻³ |
| CASP8 | casepase 8, apoptossi-related cycteine peptidase | Apoptosis, p53 signaling pathway | 9.19 × 10⁻³ |
| PDLIM5 | PDZ and LIM domain 5 | zinc ion binding | 9.46 × 10⁻³ |

TABLE 8
Comparison of the five common genes between RNA-Seq and
DASL Cancer Panel. Two independent analyses demonstrated
that 5 of the 13 genes showed significantly differential
expressions between the T1NP and T1P patients when comparing
between RNA-Seq and DASL Cancer Panel data. A high degree
of correlation was found (Spearman correlation coefficient
range from 0.82 to 0.93). logFC:log2 of fold change in
T1P samples relative to T1NP samples.

| | | | | | Spearman |
|---|---|---|---|---|---|
| Gene | RNA-Seq | | DASL Cancer Panel | | Correlation |
| Symbol | logFC | p-value | logFC | p-value | Coefficient |
| IGFBP5 | 5.28 | 2.1 × 10⁻⁴ | 1.44 | 2.3 × 10⁻² | 0.82 |
| CD44 | 4.00 | 2.0 × 10⁻² | 1.13 | 1.3 × 10⁻² | 0.93 |
| CCND1 | 3.26 | 2.4 × 10⁻² | 1.12 | 1.0 × 10⁻² | 0.86 |
| VEGF | −2.41 | 5.7 × 10⁻³ | −0.96 | 8.7 × 10⁻³ | 0.82 |
| TRAF | −2.35 | 9.7 × 10⁻³ | −0.95 | 1.4 × 10⁻² | 0.93 |

logFC:log2 of fold change in T1P samples relative to T1NP samples.

TABLE 9
List of significantly enriched biological functions
associated with T1 BCa progression.

| Ingenuity Canonical Pathways | -log(p-value) | Ratio |
|---|---|---|
| Neuregulin Signaling | 2.74E00 | 5.26E−02 |
| Small Cell Lung Cancer Signaling | 2.23E00 | 4.76E−02 |
| Notch Signaling | 2.2E00 | 7.32E−02 |
| Role of PKR in interferon induction and | 2.11E00 | 6.82E−02 |
| Antiviral Respon [symbol] | | |
| TNFR1 Signaling | 1.92E00 | 5.88E−02 |
| Metabolism of Xenobiotics by Cytochrome | 1.9E00 | 4.55E−02 |
| P450 | | |
| Integrin Signaling | 1.84E00 | 2.93E−02 |
| Amyotrophic Lateral Sclerosis Signaling | 1.76E00 | 4E−02 |
| Arachidonic Acid Metabolism | 1.73E00 | 4.04E−02 |
| Role of Tissue Factor in Cancer | 1.64E00 | 3.7E−02 |
| p38 MAPK Signaling | 1.64E00 | 3.77E−02 |
| Lysine Degradation | 1.63E00 | 5E−02 |
| N-Glycan Degradation | 1.58E00 | 8E−02 |
| Wnt/β-catenin Signaling | 1.55E00 | 2.94E−02 |
| Hereditary Breast Cancer Signaling | 1.55E00 | 3.33E−02 |
| Hypoxia Signaling in the Cardiovascular | 1.54E00 | 4.55E−02 |
| System | | |
| PI3K/AKT Signaling | 1.52E00 | 3.1E−02 |
| ILK Signaling | 1.45E00 | 2.75E−02 |
| Caveolar-mediated Endocytosis Signaling | 1.43E00 | 3.7E−02 |
| Cycling and Cell Cycle Regulation | 1.36E00 | 3.45E−02 |
| TWEAK Signaling | 1.35E00 | 5.41E−02 |
| Ovarian Cancer Signaling | 1.35E00 | 2.9E−02 |
| Aryl Hydrocarbon Receptor Signaling | 1.33E00 | 2.84E−02 |
| Xenobiotic Metabolism Signaling | 1.31E00 | 2.26E−02 |
| Cell Cycle Regulation by BTG Family | 1.31E00 | 5.56E−02 |
| Proteins | | |
| Alanine and Aspartate Metabolism | 1.26E00 | 5.41E−02 |
| Molecular Mechanisms of Cancer | 1.26E00 | 1.94E−02 |
| FAK Signaling | 1.24E00 | 3.06E−02 |
| Rank Signaling in Osteoclasts | 1.24E00 | 3.26E−02 |
| Chronic Myeloid Leukemia Signaling | 1.17E00 | 2.94E−02 |
| Melanoma Signaling | 1.17E00 | 4.55E−02 |
| Tight Junction Signaling | 1.16E00 | 2.55E−02 |
| β-alanine Metabolism | 1.1E00 | 4.35E−02 |
| HGF Signaling | 1.1E00 | 2.94E−02 |
| Fatty Acid Metabolism | 1.07E00 | 2.91E−02 |
| Rac Signaling | 1.06E00 | 2.56E−02 |
| Toll-like Receptor Signaling | 1.05E00 | 3.92E−02 |
| CD27 Signaling in Lymphocytes | 1.02E00 | 3.64E−02 |
| PTEN Signaling | 1.02E00 | 2.5E−02 |
| Fatty Acid Biosynthesis | 1.01E00 | 1E−01 |
| Ephrin Receptor Signaling | 1.01E00 | 2.06E−02 |
| Semaphorin Signaling in Neurons | 1.01E00 | 3.85E−02 |
| Endometrial Cancer Signaling | 1.01E00 | 3.64E−02 |
| Tryptophan Metabolism | 1E00 | 2.75E−02 |
| Lymphotoxin β Receptor Signaling | 9.8E−01 | 3.57E−02 |
| Cell Cycle: G1/S Checkpoint Regulation | 9.8E−01 | 3.39E−02 |
| Role of NFAT in Cardiac Hypertrophy | 9.71E−01 | 2.13E−02 |
| mTOR Signaling | 9.64E−01 | 2.16E−02 |
| Propanoate Metabolism | 9.54E−01 | 3.57E−02 |
| Cleavage and Polyadenylation of Pre-mRNA | 9.37E−01 | 8.33E−02 |
| Death Receptor Signaling | 9.29E−01 | 3.23E−02 |
| Eicosanoid Signaling | 9.29E−01 | 3.45E−02 |
| ERK/MAPK Signaling | 9.26E−01 | 2.02E−02 |
| Induction of Apoptosis by HIV1 | 9.16E−01 | 3.17E−02 |
| Taurine and Hypotaurine Metabolism | 9.04E−01 | 7.69E−02 |
| LPS/IL-1 Mediated Inhibition of RXR | 8.89E−01 | 1.98E−02 |
| Function | | |
| Linoleic Acid Metabolism | 8.81E−01 | 3.23E−02 |
| DNA Double-Strand Break Repair by | 8.74E−01 | 8.25E−02 |
| Homologous Reco [symbol] | | |
| IL-17A Signaling in Airway Cells | 8.7E−01 | 2.9E−02 |
| Angiopoietin Signaling | 8.48E−01 | 2.82E−02 |
| Agrin Interactions at Neuromuscular | 8.27E−01 | 2.99E−02 |
| Junction | | |
| Granzyme B Signaling | 8.21E−01 | 6.25E−02 |
| Extrinsic Prothrombin Activation Pathway | 8.21E−01 | 6.25E−02 |
| Sulfur Metabolism | 8.21E−01 | 6.25E−02 |
| Cardiac β-adrenergic Signaling | 8.19E−01 | 2.17E−02 |
| Macropinocytosis Signaling | 8.17E−01 | 2.63E−02 |
| Renal Cell Carcinoma Signaling | 8.07E−01 | 2.82E−02 |
| Glyoxylate and Dicarboxylate Metabolism | 7.96E−01 | 5.88E−02 |
| Cardiomyocyte Differentiation via BMP | 7.74E−01 | 5.26E−02 |
| Receptors | | |
| C21-Steroid Hormone Metabolism | 7.74E−01 | 5.56E−02 |
| Role of Macrophages, Fibroblasts and | 7.72E−01 | 1.61E−02 |
| Endothelial Cells | | |
| LPS-stimulated MAPK Signaling | 7.69E−01 | 2.53E−02 |
| Nitric Oxide Signaling in the | 7.6E−01 | 2.47E−02 |
| Cardiovascular System | | |
| Fatty Acid Elongation in Mitochondria | 7.52E−01 | 5.26E−02 |
| Maturity Onset Diabetes of Young (MODY) | 7.52E−01 | 4.76E−02 |
| Signaling | | |
| HER-2 Signaling in Breast Cancer | 7.51E−01 | 2.53E−02 |
| Regulation of elF4 and p70S6K Signaling | 7.47E−01 | 1.89E−02 |
| Role of Osteoblasts, Osteoclasts and | 7.43E−01 | 1.76E−02 |
| Chondrocytes in R [symbol] | | |
| Pantotherate and CoA Biosynthesis | 7.32E−01 | 5E−02 |
| Prostate Cancer Signaling | 7.16E−01 | 2.22E−02 |
| Phospholipid Degradation | 7.08E−01 | 2.5E−02 |
| TGF-β Signaling | 6.84E−01 | 2.25E−02 |
| Germ Cell-Sertoli Cell Junction Signaling | 6.73E−01 | 1.89E−02 |
| TR/RXR Activation | 6.69E−01 | 2.25E−02 |
| Clathrin-mediated Endocytosis Signaling | 6.67E−01 | 1.81E−02 |
| Tumoricidal Function of Hepatic Natural | 6.62E−01 | 4.17E−02 |
| Killer Cells | | |
| Factors Promoting Cardiogenesis in | 6.54E−01 | 2.2E−02 |
| Vertebrates | | |
| CDK5 Signaling | 6.54E−01 | 2.25E−02 |
| Bladder Cancer Signaling | 6.54E−01 | 2.22E−02 |
| PAK Signaling | 6.46E−01 | 1.92E−02 |
| Apoptosis Signaling | 6.46E−01 | 2.17E−02 |
| Role of JAK family kinases in IL-6-type | 6.46E−01 | 3.85E−02 |
| Cytokine Signali [symbol] | | |
| Virus Entry via Endocytic Pathways | 6.39E−01 | 2.17E−02 |
| NF-κB Signaling | 6.36E−01 | 1.76E−02 |
| Fcγ Receptor-mediated Phagocytosis in | 6.18E−01 | 2.13E−02 |
| Macrophages a [symbol] | | |
| Atherosclerosis Signaling | 6.18E−01 | 1.96E−02 |
| IL-1 Signaling | 6.18E−01 | 1.98E−02 |
| Methionine Metabolism | 6.17E−01 | 3.7E−02 |
| p53 Signaling | 6.12E−01 | 2.11E−02 |
| Glioma Signaling | 6.05E−01 | 1.89E−02 |
| IL-6 Signaling | 6.05E−01 | 2.04E−02 |
| TNFR2 Signaling | 6.03E−01 | 3.12E−02 |
| Circadian Rhythm Signaling | 5.65E−01 | 3.03E−02 |
| Selenoamino Acid Metabolism | 5.65E−01 | 3.23E−02 |
| Protein Ubiquitination Pathway | 5.63E−01 | 1.49E−02 |
| Inhibition of Angiogenesis by TSP1 | 5.42E−01 | 3.03E−02 |
| MIF-mediated Glucocortoid Regulation | 5.42E−01 | 2.7E−02 |
| Pentose and Glucoronate Interconversions | 5.42E−01 | 3.03E−02 |
| Pancreatic Adenocarcinoma Signaling | 5.38E−01 | 1.75E−02 |
| Purine Metabolism | 5.32E−01 | 1.49E−02 |
| Oncostatin M Signaling | 5.31E−01 | 2.94E−02 |
| Type I Diabetes Mellilus Signaling | 5.27E−01 | 1.75E−02 |
| Coagulation System | 5.21E−01 | 2.86E−02 |
| IL-17A Signaling in Fibroblasts | 5.21E−01 | 2.56E−02 |
| Glutamate Metabolism | 5.11E−01 | 2.78E−02 |
| CCR3 Signaling in Eosinophils | 4.95E−01 | 1.68E−02 |
| Retinol Metabolism | 4.82E−01 | 2.56E−02 |
| Antigen Presentation Pathway | 4.73E−01 | 2.33E−02 |
| Glycerophospholipid Metabolism | 4.66E−01 | 1.68E−02 |
| Role of RIG1-like Receptors in Anitviral | 4.65E−01 | 2.22E−02 |
| Innate Immunity | | |
| MIF Regulation on Innate Immunity | 4.65E−01 | 2.22E−02 |
| Thyroid Cancer Signaling | 4.48E−01 | 2.27E−02 |
| N-Glycan Biosynthesis | 4.48E−01 | 2.33E−02 |
| Synaptic Long Term Depression | 4.43E−01 | 1.55E−02 |
| GNRH Signaling | 4.43E−01 | 1.52E−02 |
| Axonal Guidance Signaling | 4.4E−01 | 1.18E−02 |
| Cdc42 Signaling | 4.21E−01 | 1.4E−02 |
| AMPK Signaling | 4.13E−01 | 1.42E−02 |
| Cardiac Hypertrophy Signaling | 4.1E−01 | 1.32E−02 |
| Mitochondrial Dysfunction | 4.09E−01 | 1.5E−02 |
| Cytotoxic T Lymphocyte-mediated Apoptosis | 4.04E−01 | 1.92E−02 |
| of Target C [symbol] | | |
| CNTF Signaling | 4.04E−01 | 1.92E−02 |
| Inositol Phosphate Metabolism | 3.97E−01 | 1.46E−02 |
| Assembly of RNA Polymerase II Complex | 3.97E−01 | 1.82E−02 |
| Amyloid Processing | 3.97E−01 | 1.92E−02 |
| Pyrimidine Metabolism | 3.93E−01 | 1.47E−02 |
| Glutathione Metabolism | 3.9E−01 | 1.96E−02 |
| Hepatic Fibrosis/Hepatic Stellate Cell | 3.9E−01 | 1.43E−02 |
| Activation | | |
| Phospholipase C Signaling | 3.89E−01 | 1.23E−02 |
| B Cell Receptor Signaling | 3.71E−01 | 1.35E−02 |
| Colorectal Cancer Metastasis Signaling | 3.69E−01 | 1.23E−02 |
| Glioblastoma Multiforme Signaling | 3.61E−01 | 1.27E−02 |
| Glutamate Receptor Signaling | 3.6E−01 | 1.64E−02 |
| Glioma Invasiveness Signaling | 3.54E−01 | 1.69E−02 |
| Mitotic Roles of Polo-Like Kinase | 3.48E−01 | 1.67E−02 |
| Myc Mediated Apoptosis Signaling | 3.48E−01 | 1.67E−02 |
| Butanoate Metabolism | 3.48E−01 | 1.72E−02 |
| Retinoic acid Mediated Apoptosis Signaling | 3.43E−01 | 1.64E−02 |
| Estrogen-Dependent Breast Cancer Signaling | 3.38E−01 | 1.54E−02 |
| ERK5 Signaling | 3.32E−01 | 1.61E−02 |
| Pyruvate Metabolism | 3.32E−01 | 1.64E−02 |
| Valine, Leucine and Isoleucine Degradation | 3.32E−01 | 1.64E−02 |
| Production of Nitric Oxide and Reactive | 3.31E−01 | 1.23E−02 |
| Oxygen Species | | |
| CD40 Signaling | 3.27E−01 | 1.47E−02 |
| GM-CSF Signaling | 3.27E−01 | 1.52E−02 |
| PXR/RXR Activation | 3.22E−01 | 1.56E−02 |
| Role of MAPK Signaling in the | 3.22E−01 | 1.59E−02 |
| Pathogenesis of Influenz [symbol] | | |
| PPARα/RXRα Activation | 3.19E−01 | 1.2E−02 |
| Non-Small Cell Lung Cancer Signaling | 3.13E−01 | 1.37E−02 |
| Starch and Sucrose Metabolism | 3.08E−01 | 1.52E−02 |
| Glucocorticoid Receptor Signaling | 3.07E−01 | 1.08E−02 |
| T Helper Cell Differentiation | 3.04E−01 | 1.45E−02 |
| IL-10 Signaling | 2.99E−01 | 1.39E−02 |
| Endothelin-1 Signaling | 2.99E−01 | 1.2E−02 |
| Acute Phase Response Signaling | 2.93E−01 | 1.16E−02 |
| Calcium Signaling | 2.93E−01 | 1.08E−02 |
| Aminosugars Metabolism | 2.9E−01 | 1.43E−02 |
| RAR Activation | 2.9E−01 | 1.16E−02 |
| IL-3 Signaling | 2.86E−01 | 1.37E−02 |
| Arginine and Proline Metabolism | 2.86E−01 | 1.41E−02 |
| Androgen and Estrogen Metabolism | 2.86E−01 | 1.41E−02 |
| IL-8 Signaling | 2.82E−01 | 1.12E−02 |
| IL-17 Signaling | 2.82E−01 | 1.35E−02 |
| BMP signaling pathway | 2.82E−01 | 1.32E−02 |
| NF-κB Activation by Viruses | 2.78E−01 | 1.27E−02 |
| LXR/RXR Activation | 2.66E−01 | 1.2E−02 |
| Dopamine Receptor Signaling | 2.66E−01 | 1.3E−02 |
| NRF2-mediated Oxidative Stress Response | 2.65E−01 | 1.06E−02 |
| Acute Myeloid Leukemia Signaling | 2.63E−01 | 1.23E−02 |
| EIF2 Signaling | 2.62E−01 | 1.06E−02 |
| VDR/RXR Activation | 2.59E−01 | 1.27E−02 |
| Ceramide Signaling | 2.59E−01 | 1.22E−02 |
| Reelin Signaling in Neurons | 2.55E−01 | 1.22E−02 |
| Regulation of Actin-based Motility by Rho | 2.45E−01 | 1.16E−02 |
| VEGF Signaling | 2.35E−01 | 1.1E−02 |
| Breast Cancer Regulation by Stathmin1 | 2.31E−01 | 1.01E−02 |
| G Beta Gamma Signaling | 2.23E−01 | 1.01E−02 |
| CTLA4 Signaling in Cytotoxic T Lymphocytes | 2.2E−01 | 1.05E−02 |
| PPAR Signaling | 2.17E−01 | 9.9E−03 |
| SAPK/JNK Signaling | 2.11E−01 | 1E−02 |
| Glycerolipid Metabolism | 2.11E−01 | 1.08E−02 |
| IGF-1 Signaling | 2E−01 | 9.8E−03 |
| Nicotinate and Nicotinamide Metabolism | 2E−01 | 1.03E−02 |

List of over-represented canonical pathways
associated with T1 BCa progression.

| Category | p-value |
|---|---|
| Cell Death | 5.34E−06-4.92E−02 |
| Cellular Growth and Proliferation | 2.99E−05-4.92E−02 |
| Cell Cycle | 3.11E−05-4.99E−02 |
| Genetic Disorder | 4.41E−05-4.99E−02 |
| Metabolic Disease | 4.41E−05-4.99E−02 |
| Cell-To-Cell Signaling and Interaction | 1.03E−04-4.99E−02 |
| Cellular Assembly and Organization | 1.03E−04-4.99E−02 |
| Tissue Development | 1.03E−04-4.82E−02 |
| Cellular Movement | 1.18E−04-4.01E−02 |
| Cardiovascular System Development and | 3.07E−04-4.99E−02 |
| Func [symbol] | |
| Embryonic Development | 3.07E−04-4.82E−02 |
| Hair and Skin Development and Function | 3.07E−04-4.82E−02 |
| Organ Development | 3.07E−04-4.82E−02 |
| Organismal Development | 3.07E−04-4.99E−02 |
| Cancer | 3.47E−04-4.92E−02 |
| Cardiovascular Disease | 3.47E−04-4.01E−02 |
| Dermatological Diseases and Conditions | 3.47E−04-4.01E−02 |
| Developmental Disorder | 3.47E−04-4.01E−02 |
| Neurological Disease | 3.47E−04-4.01E−02 |
| Cellular Development | 5.72E−04-4.99E−02 |
| Post-Translational Modification | 7.81E−04-2.03E−02 |
| DNA Replication, Recombination, and | 1.01E−03-4.01E−02 |
| Repair | |
| Immunological Disease | 1.25E−03-4.6E−02 |
| Nervous System Development and Function | 1.5E−03-1.02E−02 |
| Reproductive System Development and | 1.5E−03-3.02E−02 |
| Functi [symbol] | |
| Cell Morphology | 1.53E−03-4.01E−02 |
| Respiratory Disease | 1.87E−03-2.02E−02 |
| Reproductive System Disease | 1.98E−03-4.01E−02 |
| Tissue Morphology | 2.09E−03-4.01E−02 |
| Tumor Morphology | 2.09E−03-4.01E−02 |
| Hematological System Development and | 2.69E−03-3.02E−02 |
| Funct [symbol] | |
| Immune Cell Trafficking | 2.69E−03-1.02E−02 |
| Skeletal and Muscular Disorders | 3.59E−03-3.07E−02 |
| Gastrointestinal Disease | 4.56E−03-4.08E−02 |
| Cellular Function and Maintenance | 5.33E−03-4.67E−02 |
| Gene Expression | 5.93E−03-4.99E−02 |
| Connective Tissue Development and Function | 6.48E−03-2.68E−02 |
| Skeletal and Muscular System Development | 6.48E−03-3.02E−02 |
| an [symbol] | |
| Hematological Disease | 6.65E−03-4.6E−02 |
| Antigen Presentation | 1.02E−02-1.02E−02 |
| Carbohydrate Metabolism | 1.02E−02-4.01E−02 |
| Cell Signaling | 1.02E−02-4.99E−02 |
| Cellular Compromise | 1.02E−02-4.01E−02 |
| Connective Tissue Disorders | 1.02E−02-3.02E−02 |
| Endocrine System Development and | 1.02E−02-3.02E−02 |
| Function | |
| Hematopoiesis | 1.02E−02-3.02E−02 |
| Infectious Disease | 1.02E−02-4.32E−02 |
| Inflammatory Disease | 1.02E−02-3.02E−02 |
| Inflammatory Response | 1.02E−02-4.01E−02 |
| Lipid Metabolism | 1.02E−02-4.01E−02 |
| Lymphoid Tissue Structure and Development | 1.02E−02-1.02E−02 |
| Molecular Transport | 1.02E−02-3.7E−02 |
| Nucleic Acid Metabolism | 1.02E−02-3.02E−02 |
| Ophthalmic Disease | 1.02E−02-4.01E−02 |
| Organismal Injury and Abnormalities | 1.02E−02-2.27E−02 |
| Psychological Disorders | 1.02E−02-1.02E−02 |
| Renal and Urological Disease | 1.02E−02-4.92E−02 |
| Small Molecule Biochemistry | 1.02E−02-4.01E−02 |
| Vitamin and Mineral Metabolism | 1.02E−02-4.01E−02 |
| Drug Metabolism | 2.03E−02-4.01E−02 |
| Endocrine System Disorders | 2.03E−02-4.01E−02 |
| Hepatic System Development and Function | 2.03E−02-2.03E−02 |
| Respiratory System Development and Function | 3.02E−02-3.02E−02 |
| Antimicrobial Response | 4.01E−02-4.01E−02 |
| RNA Damage and Repair | 4.01E−02-4.99E−02 |
| RNA Post-Transcriptional Modification | 4.01E−02-4.01E−02 |
| Renal and Urological System Development | 4.01E−02-4.99E−02 |
| and [symbol] | |
| Protein Synthesis | 4.92E−02-4.92E−02 |

[symbol]

indicates data missing or illegible when filed

### REFERENCES

1. Pisani P, Parkin, D M, Bray F Ferlay, J Estimate of the worldwide mortality from 25 cancers in 1990. Int. J. Cancer 1999; 83:18-29.
2. Parkin D M, Bray F, Ferlay, J Pisani P. Global cancer statistics, 2002. CA Cancer J. Clin 2005; 55:74-108.
3. Jemal A, Siegel R, Ward E, Hao Y, Thun M J. Cancer Statistics. Cancer J Clin 2009; 55:225249.
4. Cookson M S, Hen H W, Zhang Z F et al. The natural history of high risk superficial bladder cancer: 15 year outcome. J Urol 1997; 158:62-67.
5. William S G and Stein J P. Molecular pathways in bladder cancer. Urol Res 2004; 32:373-385.
6. http://seer.cancer.gov/statfacts/html/urinb.html#survival
7. Sanchez-Carbayo M, Socci ND, Lozano J, Saint F, Cordon-Cando C. Defining molecular profiles of poor outcome in patients with invasive bladder cancer using oligonucleotide microarrays. J Clin Oncol 2006; 24:778-89.
8. Schiffer E, Vlahou A, Petrolekas A, Stravodimos K, Tauber R, Geschwend J E, et al. Prediction of muscle-invasive bladder cancer using urinary proteomics. Clin Cancer Res. 2009; 15:4935-4943.
9. Wiklund E D, Bramsen J B, Hulf T, Dyrskjφt L, Ramanathan R, Hansen T B, et al. Coordinated epigenetic repression of the miR-200 family and miR-205 in invasive bladder cancer. Int J Cancer 2011 128:1327-1334.
10. Golub T R, Slonim D K, Tamayo P, Huard C, Gaasenbeek M, Mesirov J P, et al. Molecular classification of cancer: class discovery and class prediction by gene expression monitoring. Science 1999; 286:531-537.
11. Stuart R O, Wachsman W, Berry C C, et al. In silicon dissection of cell-type-associated patterns of gene expression in prostate cancer. Proc Nail Acad Sci USA 2004; 101:615-20.
12. Blaveri E, Brewer J L, Roydasgupta R, et al. Bladder cancer stage and outcome by array-based comparative genomic hybridization. Clin Cancer Res 2005; 11:7012-22.
13. Blaveri E, Simko J P, Korkola J E, et al. Bladder cancer outcome and subtype classification by gene expression. Clin Cancer Res 2005; 11:4044-4055.
14. Dyrskjot L, Kruhoffer M, Thykjaer T, et al. Gene expression in the urinary bladder: a common carcinoma in situ gene expression signature exists disregarding histopathological classification. Cancer Res 2004; 64:4040-4048.
15. Mardis E R. The impact of next-generation sequencing technology on genetics. Trends Genet 2008; 24:133-141.
15. Wang Z, Gerstein M, Snyder M. RNA-Seq: a revolutionary tool for transcriptomics. Nat Rev Genet 2009; 10:57-63.
16. Huang Q, Lin B, Liu H, Ma X, Mo F, Yu W, et al. RNA-Seq analyses generate comprehensive transcriptomic landscape and reveal complex transcript patterns in hepatocellular carcinoma. PLoS One 2011; 6:e26168.
17. Berger M F, Levin J Z, Vijayendran K, Sivachenko A, Adiconis X, et al. Integrative analysis of the melanoma transcriptome. Genome Res 2010; 20: 413-427.
18. Roychowdhury S, Iyer M K, Robinson D R, Lonigro R J, Wu Y M, Cao X, et al. Personalized oncology through integrative high-throughput sequencing: a pilot study. Sci Transl Med 2011; 3:1-10.
19. Mostofi F K, Sobin L H, Torloni H. International histology classification of tumours, No. 10. Geneva: World Organization; 1973 Histological typing of urinary bladder tumours.
20. Edge S B, Byrd D R, Compton C C, Fritz AG, Freene F L, Trotti A, editors. American Joint Committee on Cancer (AJCC) cancer staging manual. 6^{th} ed. New York, N.Y.:Springer-Verlag; 2010.
21. Trapnell, C, Pachter L, Salzberg S L. TopHat: Discovering splice junctions with RNA-Seq. Bioinformatics 2009; 25: 1105-1111.
22. Trapnell C, Williams B A, Pertea G, Mortazavi A M, Kwan G, van Baren M J, et al. Transcript assembly and quantification by RNA-Seq reveals unannotated transcripts and isoform switching during cell differentiation. Nat Biotechnology 2010; 28: 511-515.
23. Mortazavi A, Williams B A, McCue K, Schaeffer L, Wold B. Mapping and quantifying mammalian transcriptomes by RNA-Seq. Nat Methods 2008; 5: 621-628.
24. Smyth G K. Limma: linear models for microarray data. Bioinformatics and Computational Biology Solutions using R and Bioconductor. Gentleman, R, Carey S, Dudoit R and Huber (editors). Spring, New York 2005; 397-420.
25. http://www.bioconductor.org/26. Li B, Ruotti V, Stewart R M, Thomson J A, Dewey C N. RNA-Seq gene expression estimation with read mapping uncertainty. Bioinformatics 2010; 26: 493-500.
27. Toung J M, Morley M, Li M, Cheung V G. RNA-sequence analysis of human B-cells. Genome Research 2011 June; 21(6): 991-8
28. Bibikova M, Talantov D, Chudin E, Yeakley J M, Chen J, Doucet D, et al. Quantitative gene expression profiling in formalin-fixed, paraffin-embedded tissues using universal bead arrays. Am J Pathol 2004;165:1799-1807.
29. Fan J B, Gunderson K L, Bibikova M, Yeakley J M, Chen J, Wickham Garcia E, et al. Illumina universal bead arrays. Methods Enzymol 2006;410:5773.
30. Rupp G M, Locker J. Purification and analysis of RNA from paraffin-embedded tissues. Biotechniques. 1988; 6:56-60.
31. Shujun Luo S, Khrebtukova I, Perou C, Schroth G. P. Complete RNs eq analysis of cancer transcriptomes from FFPE samples. Genome Biol. 2010; 11: sl-P35.
32. Chapman E J, Hamden P, Chambers P, et al. A comprehensive analysis of CDKN2A status in micro dissected urothelial cell carcinoma reveals potential haploinsufficiency, a high frequency of homozygous co-deletion and associations with clinical phenotype. Clin Cancer Res 2005; 11:5740-5747.
33. Oeggerli M, Tornovska S, Schram′ P, et al. E2F3 amplification and over expression is associated with invasive tumor growth and rapid minor cell proliferation in urinary bladder cancer. Oncogene 2004; 23:5616-23.
34. Feber A, Clark J, Goodwin G. et al. Amplification and overexpression of E2F3 in human bladder cancer. Oncogene 2004; 23:1627-1630.
35. Lindgren I), Frigyesi A, Gudjonsson S, Sjödahl G, Hallden C, Chebil G, et al. Combined gene expression and genomic profiling define two intrinsic molecular subtypes of urothelial carcinoma and gene signatures for molecular grading and outcome. Cancer Res. 2010; 70:3463-3472.
36. Dyrskjφt L, Kruhoffer M, Thykjaer T, et al. Gene expression in the urinary bladder: a common carcinoma in situ gene expression signature exists disregarding histopathological classification. Cancer Res 2004; 64:4040-4048.
37. Dyrskjφt L, Thykjaer T, Kruhφffer M, et al. Identifying distinct classes of bladder carcinoma using microarrays. Nat Genet 2003; 33:90-96,
38. Dyrskjφt L, Zieger K, Kruhφffer M, et al. A molecular signature in superficial bladder carcinoma predicts clinical outcome. Clin Cancer Res 2005; 11:4029-36.
39. Mor O, Nativ O, Stein A, et al, Molecular analysis of transitional cell carcinoma using cDNA microarray. Oncogene 2003; 22:7702-10,
40. Lindgren D, Liedherg F, Andersson A, et al. Molecular characterization of early-stage bladder carcinomas by expression profiles, FGFR3 mutation status, and loss of 9q. Oncogene 2006; 25:2685-2696.
41. Billerey C, Chopin I), Aubriot-Lorton M H, et al. Frequent FGFR3 mutations in apillary non-invasive bladder (pTa) tumors. Am J Pathol 2001; 158:1955-1959.
42. Wu XR. Urothelial tumorigenesis: a tale of divergent pathways. Nat Rev Cance 2005; 5:713-725.
43. Wild PJ, Herr A, Wissmann C, ct al, Gene expression profiling of progressive papillary noninvasive carcinomas of the urinary bladder. Clin Cancer Res 2005; 11:4415-4429.
44. Knowles MA. Molecular subtypes of bladder cancer: Jekyll and Hyde or chalk and cheese? Carcinogenesis 2006; 27:361-371.
45. McConkey D J, Lee, S, Choi W, Tran M, :Majewski t, Lee S et al. Molecular genetics of bladder cancer: Emerging mechanisms of tumor initiation and progression. Uro Oncology 2010 28:429-440.
46. Gui Y, Guo G, Huang Y, Hu X, Tang A, Gao S, et al. Frequent mutations of chromatin remodeling genes in transitional cell carcinoma of the bladder. Nat Genet 2011; 43:875-878.

What is claimed herein is:

1. A method of treatment comprising,
administering cystectomy or chemotherapy to a subject determined to have a level of an expression product of at least one marker gene selected from Table 1 increased relative to a reference level or a level of an expression product of at least one marker gene selected from Table 2 decreased relative to a reference level; and
not administering a cystectomy or other invasive treatment to a subject determined to have a level of an expression product of at least one marker gene selected from Table 1 not increased relative to a reference level or a level of an expression product of at least one marker gene selected from Table 2 not decreased relative to a reference level.

2. The method of claim 1, further comprising a first step of measuring, in a sample obtained from a subject in need of treatment for bladder cancer, the level of an expression product of at least one marker gene selected from Table 1 or Table 2.

3. The method of claim 1, wherein the at least one marker gene is selected from the genes of Table 3.

4. The method of claim 3, wherein the at least one marker gene comprises at least one gene selected from the group consisting of:
IGFBP5; CD44; CCND1; VEGF; TRAF4; RAB31; MDK; SNX14; ANXA1; CSPG2;
CASP8; BIRC2; PAK1; PLA2G2F; PICK1; GATA2; and ABCA5.

5. The method of claim 4, wherein the at least one marker genes comprise at least IGFBP5;
CD44; CCND1; VEGF; TRAF4; RAB31; MDK; SNX14; ANXA1; CSPG2; CASP8; BIRC2;
PAK1; PLA2G2F; PICK1; GATA2; and ABCA5.

6. The method of claim 1, wherein the at least one marker gene is selected from the group consisting of:
IGFBP5; CD44; VEGF; TRAF4; RAB31; SNX14; CASP8; PLA2G2F; PICK1; and
ABCA5.

7. The method of claim 1, wherein the at least one marker gene comprises at least two marker genes selected from the group consisting of:
IGFBP5; CD44; VEGF; TRAF4; RAB31; SNX14; CASP8; PLA2G2F; PICK1; and
ABCA5.

8. The method of claim 1, wherein the at least one marker gene comprises at least three marker genes selected from the group consisting of:
IGFBP5; CD44; VEGF; TRAF4; RAB31; SNX14; CASP8; PLA2G2F; PICK1; and
ABCA5.

9. The method of claim 1, wherein the at least one marker gene comprises at least four marker genes selected from the group consisting of:
IGFBP5; CD44; VEGF; TRAF4; RAB31; SNX14; CASP8; PLA2G2F; PICK1; and
ABCA5.

10. The method of claim 1, wherein the at least one marker gene comprises at least five marker genes selected from the group consisting of:
IGFBP5; CD44; VEGF; TRAF4; RAB31; SNX14; CASP8; PLA2G2F; PICK1; and
ABCA5.

11. The method of claim 1, wherein the at least one marker gene comprises at least six marker genes selected from the group consisting of:
IGFBP5; CD44; VEGF; TRAF4; RAB31; SNX14; CASP8; PLA2G2F; PICK1; and
ABCA5.

12. The method of claim 1, wherein the at least one marker gene comprises at least IGFBP5;
CD44; VEGF; TRAF4; RAB31; SNX14; CASP8; PLA2G2F; PICK1; and ABCA5.

13. The method of claim 1, wherein the expression products are mRNA expression products.

14. The method of claim 1, wherein the expression products are polypeptide expression products.

15. The method of claim 1, wherein the subject has TA or T1 bladder cancer.

16. The method of claim 1, wherein the sample is a tumor cell sample.

17. The method of claim 1, wherein the sample is a urine sample.

18. The method of claim 1, wherein the subject is a human.

19. A method of treatment comprising,
administering cystectomy or chemotherapy to a subject determined to have a level of an expression product of at least one gene selected from the group consisting of:
IGFBP5; ANXA1; CD44; CCND1;BIRC2; SNX14; RAB31; CASP8;
CSPG2; and PAK1;
which is increased relative to a reference level or a level of an expression product of at least one gene selected from the group consisting of:
PLA2G2F; GATA2; VEGF; TRAF4; MDK; PICK1; and ABCA5;
which is decreased relative to a reference level; and
not administering a cystectomy or other invasive treatment to a subject determined to have a level of an expression product of at leat one gene selected from the group consisting of:
IGFBP5; ANXA1; CD44; CCND1;BIRC2; SNX14; RAB31; CASP8; CSPG2; and PAK1;
which is not increased relative to a reference level or a level of an expression product of at least one gene selected from the group consisting of:
PLA2G2F; GATA2; VEGF; TRAF4; MDK; PICK1; and ABCA5;
which is not decreased relative to a reference level.

20. A method of treatment comprising,
administering cystectomy or chemotherapy to a subject determined to have a level of an expression product of at least one gene selected from the group consisting of:
IGFBP5; CD44; SNX14; RAB31; and CASP8;
which is increased relative to a reference level or a level of an expression product of at least one gene selected from the group consisting of:
PLA2G2F; VEGF; TRAF4; PICK1; and ABCA5;
which is decreased relative to a reference level; and
not administering a cystectomy or other invasive treatment to a subject determined to have a level of an expression product of at least one gene selected from the group consisting of:
IGFBP5; CD44; SNX14; RAB31; and CASP8;
which is not increased relative to a reference level or a level of an expression product of at least one gene selected from the group consisting of:
PLA2G2F; VEGF; TRAF4; PICK1; and ABCA5;
which is not decreased relative to a reference level.
